(19) United States
(12) Patent Application Publication
(10) Pub. No.: US 2008/0122428 A1
(43) Pub. Date: May 29, 2008

(54) Optimized Determination of Voltage Changes Using a Voltage-Sensitive Dye

(75) Inventors: Bernd Kuhn, Gunzburg (DE); Winfried Denk, Heidelberg (DE); Gerd Hubener, Muenchen (DE); Peter Fromherz, Munchen (DE)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignee: Max-Planck-Gesellschaft zur Forderungder Wissenschaften e. V., Munchen (DE)

(21) Appl. No.: 10/562,308

(22) PCT Filed: Jun. 25, 2004
(86) PCT No.: PCT/EP04/06916

§ 371 Date: Nov. 28, 2007

(30) Foreign Application Priority Data
Jun. 26, 2003 (EP) 03 014 601.3

(51) Int. Cl. G01N 21/64; C07D 221/18; C07D 221/04

(52) U.S. Cl. 324/96; 546/77; 546/38; 546/30; 546/26; 546/101; 546/59; 546/33; 546/28

(57) ABSTRACT

The present invention relates to a method of determining voltage changes, e.g. in cell membranes, by means of a voltage-sensitive dye.

The present invention relates to a method of determining voltage changes, e.g. in cell membranes, by means of a voltage-sensitive dye.

The recording of neuronal activity is an essential prerequisite to investigate the electric function of nerve cells, neuronal networks and the brain. Such applications basically require dyes exhibiting a great fluorescence change at small voltage changes. Numerous dyes have been developed to this end over the years (Loew, L. M.; Bonneville, G. W.; Surow, J., Biochemistry 1978, 17, 4065; Loew, L. M., Simpson, L. L., Biophys. J. 1981, 34, 353; (9) Fluhler, E., Burnham, V. G., Loew, L. M. Biochemistry 1985, 24, 5749; Grinvald, A., Hildesheim, R., Farber, I. C., Anglister, L. Biophys. J. 1982, 39, 301; Grinvald, A., Fine, A., Farber, I. C., Hildesheim, R. Biophys. J. 1983, 42, 195), especially the dyes di-4-ANEPBS and RH-421. Most popular are styrene-type hemicyanines where aniline as an electron donor is joined to pyridinium as an electron acceptor by one, two or three conjugated CC double bonds.

In conventional methods of determining voltage changes, particularly in the case of nerve cells, however, the problem is that voltage-dependent dyes typically indicate fluorescence changes of only a few percent per 100 mV membrane voltage change. Therefore, the signal-to-noise ratio is often very low due to an excitation in or including a highly efficient absorption range of the dye. Further, due to a strong phototoxicity of most photosensitive dyes only short measurement times are possible for a single cell.

An object of the present invention, therefore, was to provide a new process for an improved measurement of voltage changes overcoming the limitations of the prior art, in particular, low sensitivity and phototoxicity.

According to the invention this object is solved by a process for determining voltage changes by means of a voltage-sensitive dye, characterized in that the voltage-sensitive dye is irradiated with light having a wavelength, at which the dye has an absorption ≦20% of its absorption maximum and the fluorescence caused by irradiation with light is measured.

According to the invention changes in voltage, in particular, in transmembrane voltage, are determined using voltage-dependent dyes, which show a change in fluorescence depending on the voltage they experience. Usually lamps and filters or lasers are used for excitation. However, excitation can be effected by any light source. According to the invention excitation is effected at a wavelength or a wavelength band, at which absorption of the dye is ≦20%, preferably ≦10%, in particular, ≦5%, more preferably ≦3% and most preferably ≦2% of the absorption maximum. According to the invention no light is irradiated to the dye, which has a wavelength, at which the absorption of the dye is ≧20%, preferably ≧10%, in particular, ≧5%, more preferably ≧3% and most preferably ≧2% of its absorption maximum. Earlier it has been found with other dyes, not pure Stark-shift probes, that the sensitivity increases at the spectral wing, especially at the red spectral wing of the excitation spectrum (Loew, L. M., J. Biochem. Meth. 6 (1982) 243-260). We explored the sensitivity beyond that limited spectral range of the earlier study and with the new pure Stark-shift probes and find that we can approach the thermodynamic sensitivity limit of voltage-sensitive dyes. This method of excitation at the very red edge of the excitation spectrum can also be used with dyes that show no pure Stark-shift. The sensitivities are expected to be, higher than all previous sensitivities but lower than those of pure Stark-shift probes due to other spectrum-changing effects.

According to the invention, particularly, an increase or decrease of fluorescence is measured which can be observed in the case of the dyes when radiated with light due to a change in voltage of the environment, e.g. a change of transmembrane voltage.

Specifically, the invention relates to a method of measuring voltage changes in cells such as nerve cells and in cell membranes.

According to the invention, a voltage-sensitive dye is used. Suitable dyes are described in literature (Loew, L. M., Bonneville, G. W., Surow, J. Biochemistry 1978, 17, 4065; Loew, L. M., Simpson, L. L. Biophys. J. 1981, 34, 353; Fluhler, E., Burnham, V. G., Loew, L. M., Biochemistry 1985, 24, 5749; Grinvald, A., Hildesheim, R., Farber, I. C., Anglister, L. Biophys. J. 1982, 39, 301; Grinvald, A., Fine, A., Farber, I. C., Hildesheim, R. Biophys. J. 1983, 42, 195). Suitable fluorescent dyes, for example, are hemicyanines having a hydrophilic head group and a hydrophobic and region. The hydrophilic part usually comprises a sulfate group (negatively charged) and pyridine (positively charged), while the hydrophobic end is composed of aniline and two hydrocarbon groups. Thus, the dyes are amphiphilic and deposit in lipid membranes. In the case of these dyes the chromophore being a coherent electron system is formed by pyridine and aniline which are interconnected by alternating single and double bonds of hydrocarbons. Such dyes show an electrochromic response to membrane voltage changes, a solvatochromic effect, photoisomerism of CC double bonds and photorotamerism of CC single bonds. The electrochromic effect is due to the Stark effect. The solvatochromic effect leads to a movement of the dye in its surrounding solvent due to changes in the electric field, which also results in spectral changes.

According to the invention hemicyanine dyes having linearly anellated benzene rings are preferably used as they do not show solvatochromic effects during voltage changes and as they cannot isomerize or rotate (no CC single or CC double bonds). Preferred are those hemicyanine dyes with at least 3, in particular, at least 4, more preferred at least 5 and most preferred at least 6 and up to 20, more preferred up to 10 and most preferred up to 8 linearly anellated benzene rings. Preferred compounds have the

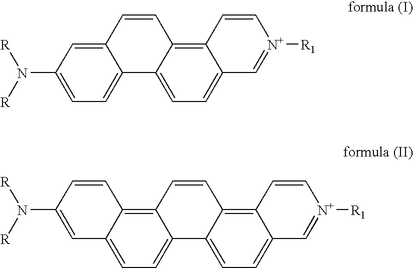

and more preferred compounds comprise the compounds ANNINE-4, ANNINE-5, ANNINE-6 and ANNINE-7 having the general formula

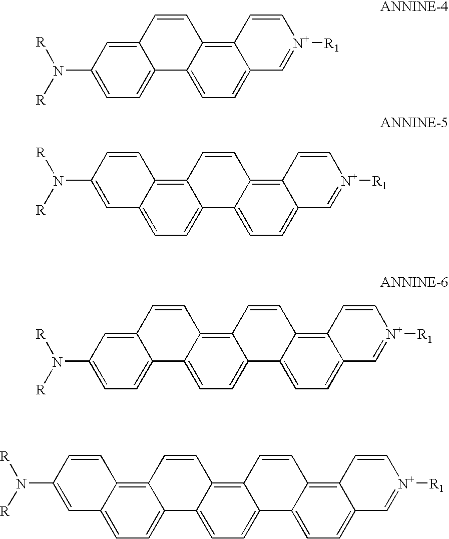

wherein each R independently is a hydrocarbon residue which can be linear or branched, saturated or mono- or polyunsatured and which has 1-30, preferably 1-10 carbon atoms, and may be substituted with hydroxyl,

### R¹ is a monovalent residue,

n is an integer from 1 to 9 and m is an integer from 0 to 8. Particularly, R¹ is an organic residue comprising 1 to 50, in particular, 1 to 30 C atoms and optionally heteroatoms, in particular, selected from N, S, O and P. While R¹ can be neutral, it is preferably a negatively or positively charged residue. R¹ preferably is a hydrocarbon residue which can be linear or branched, saturated or mono- or polyunsatured and which has 1-30, preferably 1-10 carbon atoms, e.g. methyl, ethyl, propyl, butyl, etc. Especially preferably R¹ is a residue R²—SO₃⁻, wherein R² is a hydrocarbon group which can be linear or branched and either saturated or mono- or polyunsatured and which has 1-30, particularly 1-10 carbon atoms. In a further preferred embodiment R¹ is a positively charged residue such as R²—N⁺(R)₃, e.g. (CH₂)₃N⁺(CH₃)₃, forming a dicationic dye. Such dicationic dyes show improved solubility in water up to total water solubility (without any need of additional organic solvents) while retaining voltage sensitivity. It is further preferred that R¹ is a C₁-C₁₀, in particular, a C₁-C₅ alkyl residue, for example, methyl, a phosphate residue or a hydroxyalkyl residue, for example, —(CH₂)ₙ—OH, whereby n=1-10, for example, 3. Especially preferably R is butyl residue and R¹ is a residue (CH₂)₄SO₃⁻.

Further preferred are compounds of formula (I) or (II), which have one or more substituents at the ring carbon atoms. Preferred substituents which have no influence on the chromophore are C₁-C₁₀ alkyl, especially C₁-C₅ alkyl and, particularly preferred, methyl, C₁-C₅ haloalkyl, especially CF₃ and halogen, in particular, Cl or Br. Preferably, a halogen, in particular, chlorine is introduced as a substituent at position 1 or/and 3 of the pyridine ring.

Preferred substituents having an influence on the chromophore are fluorine, aryl, O-alkyl, S-alkyl, N-alkyl₂, O-aryl, S-aryl, N-aryl₂, OH, NH₂, CO-alkyl, in particular, acetyl, ester (—O—CO— or —CO—O—) or amides, the alkyl groups each preferably having 1-10, in particular 1-5 carbon atoms and the aryl groups each preferably having 5-15, preferably 6-10 carbon atoms.

In particular, the voltage-sensitive dye is selected from ANNINE-4, ANNINE-5, ANNINE-6, ANNINE-7, ANNINE-8 (having formula (I) with n=3) or ANNINE-9 (having formula (II) with m=3) or from derivatives of those chromophores.

In particular, the dyes ANNINE-5, ANNINE-6, ANNINE-7, ANNINE-8 and ANNINE-9 show a pure electrochromic response to membrane voltage changes. This leads to a sensitivity increase at the red edge of the spectrum. Using narrowband excitation (laser) (e.g. <40 nm width) at the very long-wavelength edge of the spectrum, sensitivities of up to 70% for 100 mV can be achieved. The pure spectral shift caused by the Stark effect, which is not overlapped by other effects, e.g. solvatochromism, offers numerous advantages in practice. Sensitivity is increased and the spectra obtained are markedly easier to understand. Further, specifically ANNINE-6 and ANNINE-7 show a very large intramolecular charge shift that leads to a strong spectral shift and so to the extremely high sensitivity.

The pure spectral shift ensures that the sensitivity increases strongly at the spectral edge. This means that the signal decreases much less than the absorption cross section resulting in a high relative fluorescence change and high information obtained per absorption event. By compensation of the decreasing absorption cross section with hither excitation intensity the signal-to-noise ratio can be adapted to the needs of the experiment. Since phototoxicity is dependent on absorption, this also means that either the number of tests which can be carried out on a cell can be increased or the sensitivity of the tests can be enhanced. When increasing the excitation wavelength, a thermodynamical limit is nearly reached, at which no further sensitivity increase is achieved. In the case of ANNINE-6 and an excitation wavelength of 515 nm a sensitivity of −0.37%/mV for 1-photon excitation for dyes incorporated at the outer side of the membrane and 0.37%/mV for 1-photon excitation and 0.52%/mV for 2-photon excitation for dyes incorporated inside the cells (as expected) could be achieved. This sensitivity is four times higher than the highest hitherto published value. Such high sensitivities also enable the detection of small (e.g. ≦10 mV, particularly ≦5 mV) and/or rapid (1 ms time resolution) voltage changes. This sensitivity range is not covered by existing high throughput screening methods. The increased relative changes furthermore reduce the dependency on instrumental shortcomings, e.g. variations of the illumination intensity.

In the case of the dye ANNINE-6 measurement is carried out preferably at wavelengths in the range of from 500 nm to 530 nm.

It has been found that sensitivity (i.e. the relative fluorescence change) at wavelengths which are closer to the absorption maximum (i.e. ranges having an absorption >5%) is not so good as in the range according to the invention.

Another advantage of the ANNINE dyes preferably used according to the invention is their quick reaction, so measurements in the sub-microsecond range are possible.

As the mechanism of voltage sensitivity in ANNINEs is very simple, they show the same voltage sensitivity in cells so different as leech neurons, sea urchin eggs, zebra fish brain and HEK293 cells. Therefore, they can be used for all different cell types. This can also become important for calibration.

Further, surprisingly, the internalization and flipping of the ANNINE dyes is strongly reduced. Internalization and flipping reduces or even annihilates the voltage sensitivity of a dye. So measurement time with the ANNINE dyes is largely increased also with cells that internalize other dyes very fast (e.g. HEK293 cells).

In hemicyanine dyes with anellated benzene rings there is no photorotamerism and no photoisomerism around CC double bonds and CC single bonds. Their solvatochromism in bulk solvents suggests an intramolecular charge shift that is distinctly larger than in homologous styryl type hemicyanines. They are simple and most efficient voltage-sensitive probes of voltage transients in biological membranes. In addition the ANNINE dyes may be used as sensitive probes of polarity and local electrical fields in colloids and polymers. Strong nonlinear optical effects are also expected.

Finally, it is also to be noted that molecules are inefficiently excited by irradiation at the edge of the absorption spectrum (i.e. at ≦5% of the absorption maximum). To achieve higher excitation the intensity of the light irradiation can be increased. In this way the signal-to-noise ratio can be adjusted. In this way a longer measurement time and/or reduced damage to the tested cell can be achieved by exciting few molecules.

In another preferred embodiment of the invention voltage-sensitive dye signals were obtained with two-photon excitation. Thereby the same total energy as in the case of the above-described one-photon method is provided by two photons, i.e. the wavelength of the photons in the case of two-photon excitation is twice as long. Up to 30% higher sensitivities could be achieved at the corresponding excitation wavelengths, which allows to combine the high-voltage sensitivity of the above-described method with the advantages of two-photon excitation. The highest sensitivities that were found with 2-photon excitation are −0.52% per mV membrane-voltage change at an excitation wavelength of 1040 nm, if the dye is in the outer leaflet of the membrane (and expected +0.52%/mV, if the dye stains the inner leaflet of the membrane). The extreme red excitation using two-photon exciation allows the use of the complete emission (fluorescence) spectrum, even if the corresponding excitation wavelength based on one-photon excitation were within the emission (fluorescence) range of the dye. Two-photon excitation especially offers the advantages of deep penetration into the tissue, low phototoxicity because absorption takes place only in the focus as well as low background fluorescence. Due to this the method can be performed even more reliably.

The invention further relates to a voltage-sensitive dye having the formula (I)

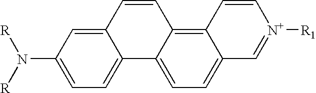

or formula (II)

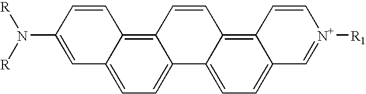

wherein each R independently is a hydrocarbon residue which optionally can be substituted with hydroxyl, R¹ is a monovalent residue, n is an integer from 1 to 9 and m is an integer from 0 to 8, which compounds optionally can have one or more substituents at ring carbon atoms. Preferred compounds are as given herein above. In a preferred embodiment, the invention relates to such dyes with the proviso that the compound is not ANNINE-5 or ANNINE-6.

The invention is further illustrated by the enclosed Figures and the Examples given below.

### EXAMPLE 1

### Synthesis of ANNINE Dyes

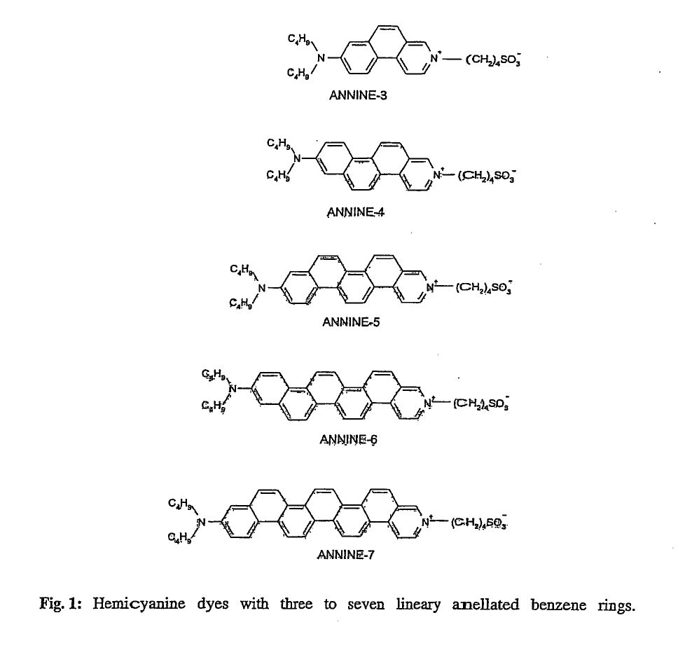

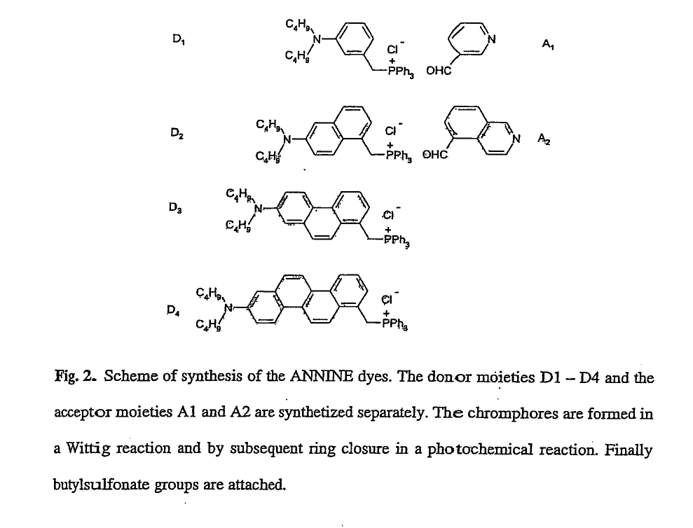

The ANNINE dyes were assembled by combining four different donor moieties (D₁-D₄) with two different acceptor moieties (A₁-A₂) as illustrated in FIG. 2.

3-N,N-Dibutylaminobutyl benzoate (1)²⁰. 1-Iodobutane (114.3 ml, 1 mol) was added to a mixture of 3-aminobenzoic acid (Fluka, Switzerland, 34.3 g, 250 mmol) in 250 ml DMF and K₂CO₃ (103.5 g, 750 mmol). The mixture was stirred at 100° C. overnight and distributed between EtOAc and water. The organic layer was washed with brine, dried (Na₂SO₄) and concentrated in vacuo. Pure 1 (64.9 g, 85% yield, colourless oil) was obtained by distillation (bp 153° C., 0.01 mbar).

3-N,N-Dibutylaminobenzyl alcohol (2). To a magnetically stirred mixture of LiAlH₄ (5.3 g, 140 mmol) in 200 ml of anhydrous diethyl ether was added dropwise a solution of 1 (74.1 g, 240 mmol) in 200 ml anhydrous diethyl ether. The mixture was stirred overnight, then quenched by addition of ice cold water, triturated with 40% NaOH and the organic layer separated. The ether extract was washed with brine, dried (Na₂SO₄) and evaporated. Destillation provided 2 (oil, bp 105-110° C., 0.01 mbar, 91% yield).

3-N,N-Dibutylaminobenzyl chloride (3). PCl₅ (46.0 g, 220 mmol) was added in portions to 2 (52.3 g, 220 mmol). The black viscous mixture was stirred at 100° C. for 1 h and then at room temperature overnight. The reaction mixture was quenched with ice and diluted ammonia and extracted with ether. The extract was washed with brine, dried (Na₂SO₄) and the solvent was evaporated. Chromatography (SiO₂, EtOAc:heptane 18:5) provided pure 3 (oil, bp 95° C., 0.01 mbar, yield 79%).

3-N,N-Dibutylaminobenzyltriphenylphosphonium chloride (4). A solution of 3 (19.4 g, 152 mmol) and PPh₃ (40 g, 152 mmol) in 120 ml toluene was refluxed for 16 h at 110° C. Upon cooling the white precipitate was collected, washed with Et₂O and dried for 24 h at 50° C. in vacuo to yield pure 4 (mp 165° C., yield 63%).

1-Bromo-6-N,N-dibutylaminonaphthalene (5). The synthesis was performed similarly to that previously described for 6-bromo-2-N,N-dibutylaminonaphthalene²⁰. A mixture of 6-amino-1-bromonaphthalene²¹ (29.15 g, 131.2 mmol, obtained from 2-nitronaphthalene (Lancaster, U.K.), via 1-bromo-6-nitronaphthalene^{21,22} and subsequent reduction), 1-iodobutane (52.5 ml, 492.2 mmol) and anhydrous K₂CO₃ (36.0 g, 260 mmol) was heated in 250 ml dry DMF at 130° C. for 48 h. Upon cooling water was added and the mixture extracted with CHCl₃ several times. The organic extracts were dried (Na₂SO₄) and the solvent evaporated. By chromatography (SiO₂, EtOAc: heptane 3:7) pure 5 (brown oil, yield 77%) was obtained.

6-N,N-Dibutylamino-1-cyanonaphthalene (6)²³. A mixture of 5 (3.3 g, 10 mmol) and CuCN (1.07 g, 12 mmol) and 10 ml dry pyridine were refluxed at 220° C. for 72 h. Upon cooling and evaporation of the solvent the compound 6 (yellow solid, mp 33° C., yield 78%) was isolated by chromatography (SiO₂ EtOAc:heptane 3:7).

6-N,N-Dibutylaminonaphthalene-1-carboxylic acid (7). A solution of 6 (1.4 g, 5 mmol) and NaOH (0.3 g, 7.5 mmol) in 20 ml 1-pentanole was stirred at 170° C. for 72 h. Then the solvent was evaporated and the residue distributed between EtOAc and water and acidified with 1 N HCl to pH 6. The organic layer was separated and the water extracted twice. The combined organic layer was dried (Na₂SO₄) and evaporated to give 7 (yellow solid, mp 78° C., quant.).

6-N,N-Dibutylamino-1-hydroxymethylnaphthalene (8)²⁴. To a stirred and ice-cooled mixture of LiAlH₄ (0.6 g, 15.8 mmol) and dry diethylether (50 ml) a solution of 7 (5.6 g, 18.7 mmol) in 70 ml dry diethylether was added dropwise; then the cooling was removed and the mixture stirred overnight. After the addition of water the resulting precipitate was removed by filtration; then the organic layer was washed with brine and dried (Na₂SO₄) and the solvent was evaporated. The resulting compound 8 (oil, yield 90%) still containing minor impurities was used for the next step. The direct synthesis of 8 starting from 5 via the lithiated intermediate and subsequent reaction with formaldehyde is also possible but less effective.

1-Bromomethyl-6-N,N-dibutylaminonaphthalene (9)²⁴. To a solution of the alcohol 8 (14.5 g, 51 mmol) in 60 ml of toluene PBr₃ (2.4 ml, 26 mmol) was added under vigorous stirring. After the reaction mixture was stirred overnight, icy-cold water and then 10% NaHCO₃ was added. The product was extracted with EtOAc, the organic layer washed with brine, dried (Na₂SO₄) and the solvent evaporated. After purification by column chromatography (SiO₂, EtOAc:heptane 1:4) 13.2 g 9 (yellow oil, which solidifies in the freezer, yield 75%) were obtained.

6-N,N-Dibutylaminonaphthyl-(10)-methyltriphenylphosphoniumbromide (10). A solution of 9 (12.7 g. 36.6 mmol) and PPh₃ (9.6 g, 36.6 mmol) in 40 ml was refluxed for 7 h. Upon cooling 450 ml diethylether were added and the precipitate washed with diethylether twice to give pure 10 (22 g, mp 185-188° C., yield 99%).

2-Methoxymethylbenzaldehyde (11). 1,2-Bis(hydroxymethyl)benzene (Lancaster, U.K.) was reacted to 2-methylmethoxybenzyl alcohol (1 equiv. NaH in THF, then MeI, oil, yield 50%); subsequent oxidation (MnO₂ in CHCl₃) resulted 11 (oil bp 52° C. (0.012 mbar), yield 82%)²⁵.

7-N,N-Dibutylaminophenanthryl-(1)-methyltriphenylphosphoniumbromnide (12) resp. 8-N,N-dibutylaminochrysenyl-(1)-methyltriphenylphosphoniumbromide (13). 20 mMol of the phosphonium salt 4 resp. 10 and 20 mMol sodiummethoxide were dissolved in dry methanol and stirred at 60° C. for 3 h. Then 20 mMol of the aldehyde 11 in MeOH were added dropwise and the solution was allowed to reflux overnight. After evaporation of the solvent the crude product was purified by chromatography (silicagel; EtOAc:heptane 1:5); the obtained alkenes E/Z-2-(3-N,N-dibutylamino-styryl)benzylmethylether 14a (yield 50%) resp. E/Z-2-(6-N,N-dibutylamino-1-naphthyl)vinylbenzylmethylether 15a (yield 61%) were cyclizysed as described below (20 h, solvent CH₂Cl₂) to the 7-N,N-dibutylamino-1-methoxy-methylphenanthrene 14 (yield 40%, oil) resp. 2-N,N-dibutylamino-7-methoxymethylchrysene 15 (yield 50%, m.p. 215° C.). 10 mMol of the ethers 14 resp. 15 were stirred with 20 ml 62% HBr at 60° C. for 10 min. After neutralisation (KHCO₃) the crude products were extracted with CHCl₃ and purified by chromatography (silicagel; EtOAc:heptane 1:5) to receive 7-N,N-dibutylamino-1-bromomethylphenanthrene 16 (yield 53%, oil) resp. 2-N,N-dibutylamino-7-bromomethylchrysene 17 (yield 50%, m.p. 186° C. (dec.)). Treatment of 16 resp. 17 with triphenylphosphine yielded the phosphonium salts 12 (yield 81%, m.p. 213-214° C.) resp. 13 (yield 93%, mp 206° C.).

5-Formylisoquinoline (18) was obtained from 5-bromoisoquinoline²⁶ in analogy to 4-formylisoquinoline²⁷ (mp 116° C.²⁸, yield 55%).

E/Z-3-(3-N,N-Dibutylaminostyryl)-pyridine (19): 3.5 mMol of the phosphonium salt 4 was dissolved in dry MeOH, 190 mg (3.5 mMol) NaOMe was added and the mixture was allowed to reflux for 3 h; then 3.5 mMol of the 3-formylpyridine (Fluka) dissolved in MeOH was added; then the solution was refluxed overnight. The E/Z mixture was isolated by flash chromatography (silicagel, EtOAc:heptane 1:1). Yield 28%, yellow oil; EIMS m/z 308 (M⁺), 265 (M⁺-CH₇, C₂₁H₂₈N₂ requires 308.2).

E/Z-3-(6-N,N-Dibutylamino-1-naphthyl)-vinylpyridine (20): Same procedure as described for 19, but using the phosphonium salt 10. Yield 85%, yellow oil; EIMS m/z 358 (M⁺), 315 (M⁺-C₃H₇, C₂₅H₃₀N₂ requires 358.2).

E-5-(6-N,N-Dibutylamino-1-naphthyl)-vinylisoquinoline (21): Same procedure as described for 19, but using the phosphonium salt 10 and aldehyde 18. Yield 73%, yellow solid, mp 128-130° C. (EtOAc); EIMS m/z 408 (M⁺), 365 (M⁺-C₃H₇, C₂₉H₂₈N₂ requires 408.3).

E/Z-5-(7-N,N-Dibutylamino-1-phenanthryl)-vinylisoquinoline (22): Same procedure as described for 19, but using the phosphonium salt 12 and aldehyde 18. Yield 82%, yellow solid, EIMS m/z 458 (M⁺), 415 (M⁺-CH₇, C₃₃H₃₄N₂ requires 458.3).

E/Z-5-(8-N,N-Dibutylamino-1-chrysenyl)-vinylisoquinoline (23): Same procedure as described for 19, but using the phosphonium salt 13 and aldehyde 18. Yield 80%, yellow solid; FABMS m/z 509.3 (M+1, C₃₇H₃₆N₂ requires 508.3).

Cyclisation of the alkenes 19-23²⁹: A 5-10⁻³ molar solution of the corresponding E- or E/Z-alkene was irradiated (45 min-40 h, duran glass, room temperature, CH₂Cl₂, unless stated otherwise) by a Hg high pressure lamp (TQ 150, Heraeus-Noblelight, Germany); after evaporation of the solvent the cyclisation products 24-26 were isolated by flash chromatography (EtOAc:heptane 1:1); crude 27 and 28 were obtained by precipitation from CHCl₃/MeOH and used for the final step without further purification.

7-N,N-Dibutylamino-2-azaphenanthrene (24): yield 48%, 45 min irradiation (quartz glass, −30° C., 2-methyltetrahydrofuran), yellow oil; FABMS m/z 307.2 (M+1, C₂₁H₂₆N₂ requires 306.2).

8-N,N-Dibutylamino-2-azachrysene (25): yield 22%, 45 min irradiation (quartz glass, 0° C., cyclohexane), pale yellow solid, m.p. 213° C. (EtOAc); EIMS m/z 356 (M⁺), 313 (M⁺-C₃H₇, C₂₅H₂₈N₂ requires 356.2).

10-N,N-Dibutylamino-3-azapicene (26): yield 25%, 40 h irradiation (cyclohexane), pale yellow solid (EtOAc), still containing minor impurities; FABMS m/z 407.2 (M+1, C₂₉H₃₀N₂ requires 406.2).

11-N,N-Dibutylaminobenzo[m]-3-azapicene (27): yield 40% (crude product), 15 h irradiation; EIMS m/z 456 (M⁺), 413 (M⁺-C₃H₇, C₃₃H₃₂N₂ requires 456.3).

12-N,N-Dibutylaminonaphtho[5,6-m]-3-azapicene (28): yield 46% (crude product), 15 h irradiation; EIMS m/z 506 (M⁺), 463 (M⁺-C₃H₇, C₃₇H₃₄N₂ requires 506.3).

ANNINES 3-7: A mixture of 2 mMol of 24-28 and 5.4 g (40 mMol) 1,4-butane sultone was stirred at 120° C. for 4 h²⁰. The resulting precipitate is collected, washed with ether and purified by flash chromatography (silica gel, MeOH or CH₂Cl₂:MeOH:H₂O 50:20:4 and Sephadex LH 20 (Pharmacia), MeOH) and recrystallised.

ANNINE-3: yield 24%, yellow solid m.p. >300° C. (MeOH/heptane); FABMS m/z 443.2 (M+1, C₂₅H₃₄N₂O₃S requires 442.2).

ANNINE-4: yield 35%, yellow solid m.p. >300° C. (MeOH); FABMS m/z 493.5 (M+1, C₂₉H₃₆N₂O₃S requires 492.2).

ANNINE-5: yield 40%, red-orange solid m.p. >300° C. (MeOH); FABMS m/z 543.3 (M+1, C₃₃H₃₈N₂O₃S requires 542.3).

ANNINE-6: yield 21%, red-orange solid m.p. >300° C. (MeOH); FABMS m/z 593.4 (M+1, C₃₇H₄₀N₂O₃S requires 592.3).

ANNINE-7: yield 10%, red solid m.p. >300° C. (MeOH); FABMS m/z 643.5 (M+1, C₄₁H₄₂N₂O₃S requires 642.3).

The structure of all dyes and intermediates was confirmed by mass spectroscopy and proton NMR.

The triphenylphosphonium salt D₁ was obtained in four steps from 3-aminobenzoic acid via 3-N,N-dibutylaminobutyl benzoate (1-iodobutane and K₂CO₃)²⁰, 3-N,N-dibutylaminobenzyl alcohol (LiAlH₄), 3-N,N-butylaminobenzyl chloride (with PCl₅) and subsequent treatment with PPh₃. This method is superior to the synthesis using 3-aminobenzaldehyde dimethyl acetal as a starting material¹⁸. For the synthesis of D₂ we brominated 2-nitronaphthalene; the resulting 1-bromo-6-nitronaphthalene^{21,22} was reduced to 6-amino-1-bromonaphthalene (SnCl₂, HCl). Alkylation with 1-iodobutane leads to 1-bromo-6-N,N-dibutylaminonaphthalene²⁰. Via 6-N,N-dibutylamino-1-cyanonaphthalene²³ (CuCN) and 6-N,N-dibutylaminonaphthalene-1-carboxylic acid (NaOH) we obtained by reduction (LiAlH₄) 6-N,N-dibutylamino-1-hydroxymethylnaphthalene²⁴. (A direct synthesis starting from 1-bromo-6-N,N-dibutylaminonaphthalene via the lithiated intermediate and subsequent reaction with formaldehyde was less effective.) After bromination (PBr₃)²⁴ of the alcohol, a treatment with PPh₃ resulted in D₂.

To obtain D₃ we joined D₁ to 2-methoxymethylbenzaldehyde²⁵ by a Wittig reaction. Photocyclization of the alkenes E/Z-2-(3-N,N-dibutylamino-styryl) benzylmethylether produced 7-N,N-dibutylamino-1-methoxymethylphenanthrene. By ether cleavage we obtained 7-N,N-dibutylamino-1-bromomethylphenanthrene and with triphenylphosphine the product D₃. Starting from D₂ the same reaction sequence was performed to obtain D₄.

The acceptor moiety A₁ was commercially available. A₂ was obtained from 5-bromoisoquinoline²⁶ (BuLi, then DMF)^{27,28}. Proper donor and acceptor moieties were chosen for Wittig reactions and subsequent photocyclization²⁹ to build up the scaffold of the ANNINES. So for example D₃ and A₂ were used for the formation of the six anellated rings of ANNINE-6. The last synthetic step was the reaction with 1,4-butane sultone.

### REFERENCES OF EXAMPLE 1

(20) Hassner, A.; Birnbaum, D.; Loew, L. M. J. Org. Chem. 1984, 49, 25-46.
(21) v. Braun, J.; Hahn, E.; Seemann, J. Chem. Ber. 1922, 55, 1687.
(22) Fieser, L. F.; Riegel, B. J. Am. Chem. Soc. 1937, 59, 2561.
(23) Newman, M. S. J. Am. Chem. Soc. 1937, 59, 2472.
(24) Szmuszkovicz, J.; Bergmann, E. D. J. Am. Chem. Soc. 1953, 75, 353.
(25) Kirmse, W.; Kund, K. J. Am. Chem. Soc. 1989, 111, 1465.
(26) Osborn, A. R.; Schofield, L. N. J. Chem. Soc. 1956, 4191.
(27) Sainsbury, M.; Brown, D. W.; Dyke, S. F.; Clipperton, R. D. J.; Tonkyn, W. R. Tetrahedron 1970, 26, 2239.
(28) Rodionov, V. M.; Alekseeva E. N.; Vleduts G. J. Gen. Chem. USSR 1957, 27, 809.
(29) Muszkat, K. A. Top. Curr. Chem. 1980, 88, 91.

### EXAMPLE 2

### Absorption and Fluorescence Spectra

Solutions. We studied the absorption and fluorescence spectra in 17 solvents of increasing polarity: chloroform, decanol, benzyl alcohol, dichloromethane, octanol, cyclohexanol, hexanol, pentanol, cyclopentanol, butanol, propanol, ethylene glycol, dimethylformamide, acetone, ethanol, acetonitrile and methanol. Solvents of highest available purity were used (Merck, Darmstadt and Sigma, Taufkirchen, Germany). Stock solutions were made from methanol and chloroform at a volume ratio of 1:2 at a concentration of 1 mM for ANNINE-3, ANNINE-4 and ANNINE-5, and due to lower solubility 200 μM for ANNINE-6 and ANNINE-7. These stock solutions were diluted by the different solvents to a final concentration 5 μM, with the exception of ANNINE-7 in pentanol, butanol, acetone and acetonitrile where a concentration of 1 μM was used.

Spectra. Absorption spectra were measured with a Varian Cary 3E spectrometer (Varian, Mulgrave, Victoria, Australia) at room temperature. Emission spectra were measured with a SLM Aminco 8100 fluorescence spectrometer that was adapted to the long wavelength region with a Spectra Pro-275 monochromator from Acton Research (Acton, Mass., USA) containing appropriate gratings for the wavelength region up to 1200 nm and an avalanche photodiode as a detector (Polytec, Waldbronn, Germany). The spectrometer was calibrated with a 45 Watt quartz-halogen tungsten coiled filament lamp (OL 245M, Optronic Laboratories, Orlando, USA) as a standard of spectral irradiance. Calibration and measurement of the spectra were performed under magic angle conditions³⁰. For ANNINE-3 the excitation bandwidth was 8 nm and the emission bandwidth was 18 nm. For ANNINE-4, ANNINE-5, ANNINE-6 and ANNINE-7 we used an excitation bandwidth of 16 nm and an emission bandwidth of 18 nm.

Evaluation of maxima: The maxima of the absorption spectra were automatically calculated by the Varian Cary software. For evaluation of the fluorescence maxima we fitted a log normal curve to each spectrum³¹ according to Eq. 1 with an amplitude F _{ν̅}^{MAX} a spectral maximum ν̅^{MAX}, a spectral width W and a spectral asymmetry b, excluding the contribution of the S₀/S₂ transition.

$${F}_{\overline{v}} ( \overline{v} ) = {F}_{\overline{v}}^{MAX} exp \{ - \frac{{ln}^{2} [ 1 + 2 b ( \overline{v} - {v}^{\overline{M} AX} ) / W ]}{{b}^{2} / ln 2} \} \qquad ( 1 )$$

The maximum was taken from the fit. For those solvents, where the second order Rayleigh scattering peak of the excitation light appeared on the flank of the spectrum, only the region of the maximum was used for evaluation.

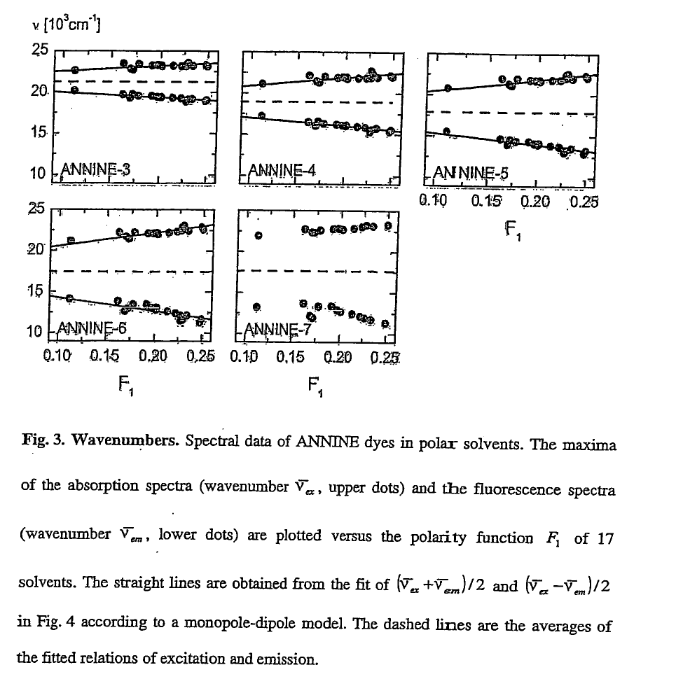

Solvatochromism. The wavenumbers ν̅_{abs} and ν̅ₑₘ of the maxima of absorption and emission of the five hemicyanine dyes ANNINE-3 to ANNINE-7 in 17 solvents are plotted in FIG. 3. The solvents are characterized by the polarity function F₁(∈,n) defined by Eq. 2³² that depends on the static relative dielectric constant ∈, the refractive index n of the solvent and an intramolecular dielectric constant ∈ᵢ.

$${F}_{1} ( ɛ , n ) = \frac{1}{{ɛ}_{i}} \cdot ( \frac{{ɛ}_{i} - {n}^{2}}{{ɛ}_{i} + 2 {n}^{2}} - \frac{{ɛ}_{i} - ɛ}{{ɛ}_{i} + 2 ɛ} ) \qquad ( 2 )$$

The values of F₁(∈,n) with ∈ᵢ=2 are for chloroform 0.1120, decanol 0.1623, benzyl alcohol 0.1697, dichloromethane 0.1722, octanol 0.1779, cyclohexanol 0.1915, hexanol 0.1971, pentanol 0.2011, cyclopentanol 0.2016, butanol 0.2136, propanol 0.2223, ethylene glycol 0.2272, dimethylformamide 0.2273, acetone 0.2294, ethanol 0.2342, acetonitrile 0.2479 and methanol 0.2497.

With all dyes there is a common trend for the maxima of absorption and fluorescence: increasing polarity shifts the absorption spectra to the blue and the emission spectra to the red. For each dye that divergent solvatochromism is rather symmetrical over the whole range of accessible polarity. I.e. it seems that the solvatochromism can be assigned to an increasing Stokes shift ν̅_{abs}− ν̅ₑₘ with an invariant average ( ν̅_{abs}+ ν̅ₑₘ)/2. I

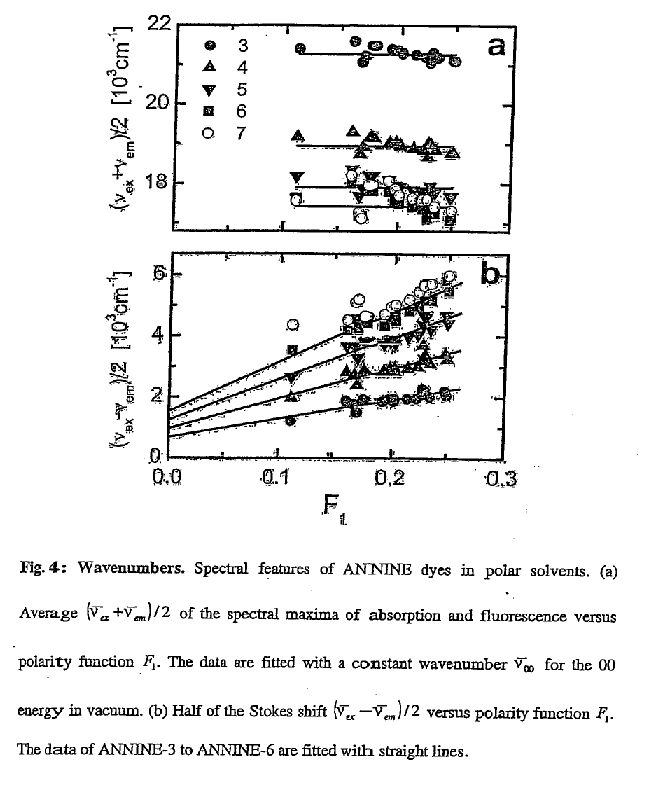

Solvatochromism. From the experimental wavenumbers of absorption and emission in FIG. 3 we evaluate the averages ( ν̅ₑₓ+ ν̅ₑₘ)/2 and differences ( ν̅ₑₓ− ν̅ₑₘ)/2 and plot them versus F₁(E,n). As shown in FIGS. 4a and 4b, the values of ( ν̅ₑₓ+ ν̅ₑₘ)/2 vary only by a few percent over the whole range of polarity, whereas the values of ( ν̅ₑₓ− ν̅ₑₘ)/2 increase rather linearly.

Electrochromism. When a dye with an intramolecular charge displacement δ_{EG} is placed in an external electrical field, its absorption and emission band are shifted by a linear molecular Stark effect. In a biological membrane, the change of the electrical field is given by the change of membrane voltage ΔV_{M} and by the membrane thickness d_{M}.

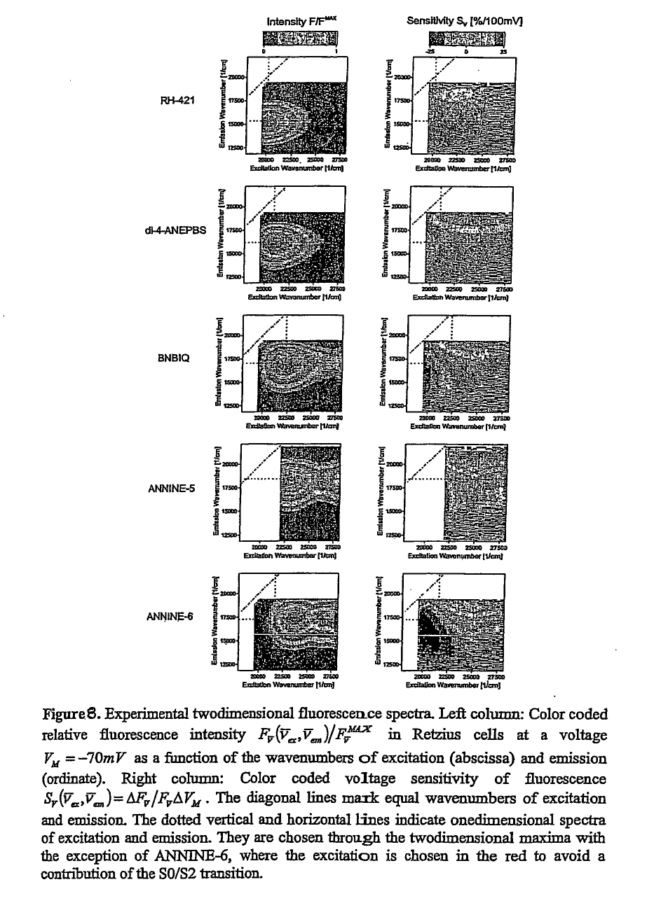

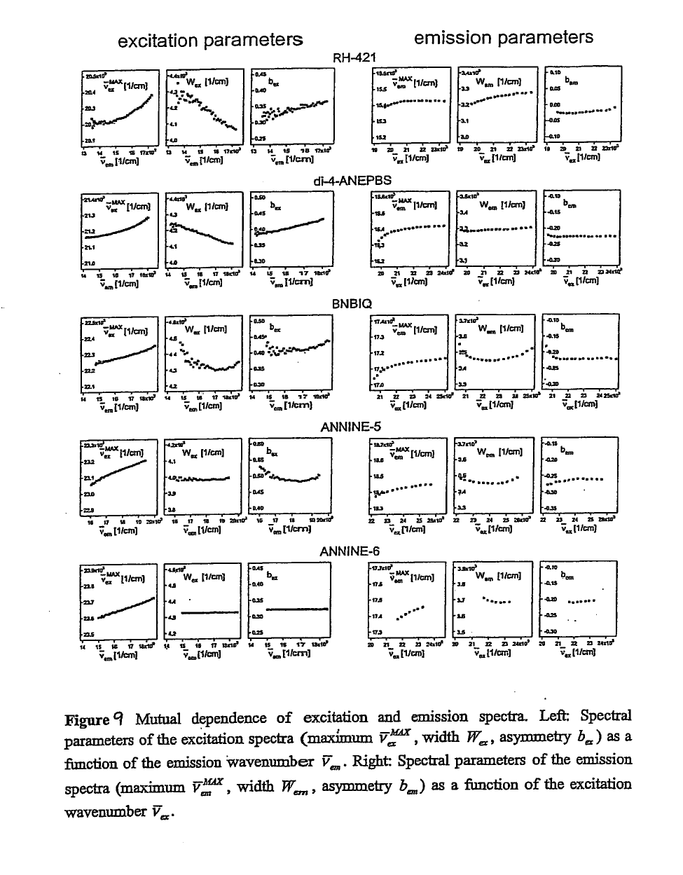

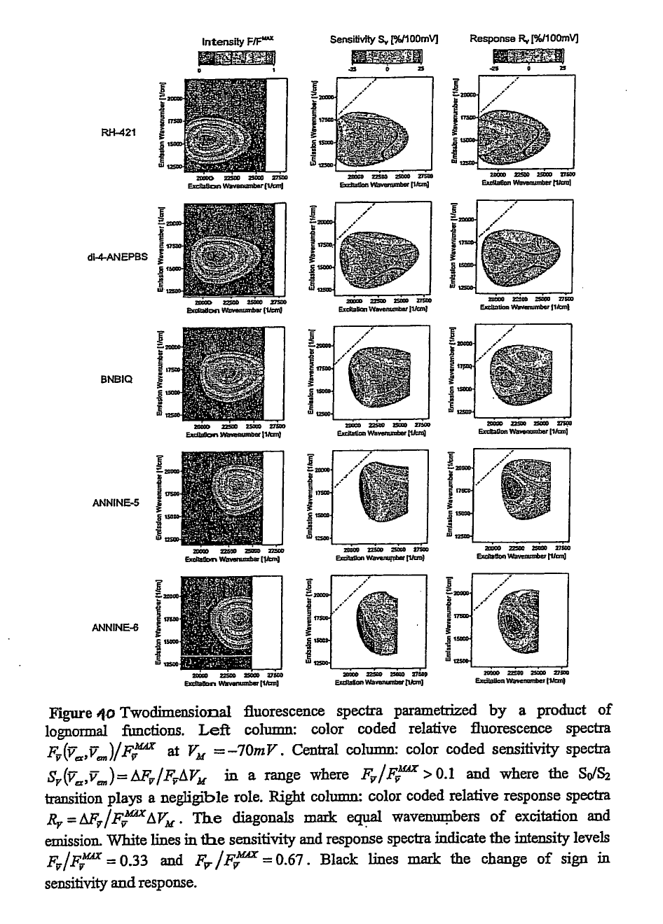

We plot the expected electrochromic shift Δ ν̅_{electro} in a membrane of thickness d_{M}=4 nm and a voltage change ΔV_{M}=100 mV versus the solvatochromic sensitivity Δ ν̅ₛₒₗᵥ in FIG. 6 for three different radii a=5, 7, 9 A. assuming a perfect orientation cos θ=1.

The experimental solvatochromic sensitivities Δ ν̅ₛₒₗᵥ of the ANNINE dyes are marked by vertical lines in FIG. 6. The experimental electrochromic shifts of ANNINE-5 and ANNINE-6 in a neuron membrane are indicated by dots.¹⁹

The electrical response in the neuron are in a range predicted by solvatochromism. Yet, the increase of electrochromism from ANNINE-5 to ANNINE-6 is stronger than expected for constant orientation cos θ in the membrane and constant effective radius a in bulk solvents.

### EXAMPLE 3

The voltage sensitivity of the anellated hemicyanines ANNINE-6 and ANNINE-5 and also of BNBIQ, di-4-ANEPBS and RH-421 was investigated. Two-dimensional fluorescence spectra of excitation and emission are measured in leech neurons at defined membrane voltages. The voltage induced changes of fluorescence are parametrized in terms of changes of spectral parameters. ANNINE dyes are far more voltage sensitive than the classical styryl dyes and their sensitivity can be assigned almost completely to an identical spectral shift of excitation and emission that is caused by a molecular Stark effect.

Dyes. The styryl dye RH-421 [11] was obtained from Molecular Probes (Eugene, Oreg., USA). The styryl dye di-4-ANEPBS [9] and the biaryl dye BNBIQ [23] were synthetized by Gerd Hübener. The synthesis of the dyes ANNINE-5 and ANNINE-6 is described above.

Neurons. Ganglia of the leech Hirudo medicinalis (Moser, Schorndorf, Germany) were dissected and pinned on a Sylgard coated dish in Leibowitz-15 medium (L-5520, Sigma, Deisenhofen) with 5 mg/ml glucose, 0.3 mg/ml glutamine and 3 μg/ml gentamycin sulfate (G-3632, Sigma) [24]. After opening the tissue capsules the ganglia were incubated in dispase/collagenase (Boehringer, Mannheim, 2 mg/ml L-15 medium) for 1 hour at room temperature. Retzius cells (soma diameter 60-90 μm) were dissociated by aspiration into a firepolished micropipette and washed with Leibowitz-15 medium [24]. The cells were seeded on an uncoated glass cover slip in a silicone chamber (Flexiperm-mikro 12, Vivascience AG, Hannover, Germany) with Leibowitz-15 medium and 2.5% fetal bovine serum (10106, Gibco, Eggenstein) and kept for one or two days at 20° C.

Staining. The dyes were solubilized in Leibowitz-15 medium with sodium cholate (Sigma, St. Luise, USA) [25]. We used 4.3 mM RH-421 with 10 mM cholate, 4.3 mM di-4-ANEPBS with 10 mM cholate, 10 mM BNBIQ with 10 mM cholate, 1 mM ANNINE-5 with 23 mM cholate and 1 mM ANNINE-6 with 25 mM cholate. After centrifugation, the staining solutions were added to the culture chambers with the neurons at a volume ratio of 1:1000 30 min before patching. The solubility of the dyes in water decreases in the series RH-421, di4-ANEPBS, BNBIQ, ANNINE-5 and ANNINE-6. In correspondence, the fluorescence intensity of cells stained with RH-421 and di-4-ANEPBS dropped after exchanging the medium by a dye-free solution due to desorption from the cells, whereas the intensity with ANNINE-5 and ANNINE-6 remained constant for at least 1.5 h. With respect to bleaching and phototoxicity, we did not observe significant differences under comparable conditions of staining and illumination.

Electrophysiology. Patch pipettes with a tip diameter of 5-10 μm were made from micro haematocrite tubes (Assistent, Karl Hecht, Sondheim/Rhön, Germany) using an all-purpose puller (DMZ-Universal Puller, Zeitz-Instrumente, Augsburg, Germany). They were filled with 140 mM KCl, 1.5 mM MgCl₂, 10 mM Hepes and 10 mM EGTA, pH 7.3. The resistance of the pipettes was around 0.4 MΩ. The pipettes with Ag/AgCl electrodes were connected to a single electrode patch-clamp amplifier (SEC-10L, npi, Tamm, Germany). The patching of Retzius cells lead to a seal resistance of about 500 MΩ. Whole cell contact [26] was achieved by breaking the membrane by suction using a water-jet vacuum pump. The bath was held at ground potential with a Ag/AgCl electrode. We kept the cells at an intracellular voltage of V_{M}=−40 mV. The fluorescence spectra were measured at a hyperpolarized voltage of V_{M}=−70 mV and a depolarized voltage of about V_{M}=10 mV.

Optical setup. The spectrometer is sketched in FIG. 2. It is built on the basis of an inverted microscope (Axiovert 35, Zeiss, Oberkochen, Germany) with a high numerical aperture oil immersion objective (Neofluar 100×/1.3 oil). The light of a 75 W Xenon short arc lamp (Ushio, Hyogo, Japan) is spectrally resolved (resolution 16 nm) by a grating monochromator (J&M, Aalen, Germany). It is fed into the microscope with an optical multimode fibre (diameter 1.2 mm) and collimating optics (J&M). Nerve cell are illuminated through a dichroic mirror (AHF Analysentechnik, Tübingen, Germany) with a splitting wavelength of 520 nm for RH-421, di-4-ANEPPS, BNBIQ and ANNINE-6 and of 460 nm for ANNINE-5. The illumination is controlled by a shutter and a field diaphragm. The light emitted by the stained cell is collected by the objective. It passes through the dichroic mirror and a long pass filter (AHF Analysentechnik, Tübingen) with cutoff at 520 nm for RH-421, di-4-ANEPBS, BNBIQ and ANNINE-6 and at 470 nm for ANNINE-5 and is guided by an optical fibre (0.6 mm) to a diode array spectrometer (J&M) with a spectral range from 307 nm to 1135 nm at a resolution of 3.1 nm.

Calibrated fluorescence spectra. In order to derive molecular parameters from the experimental data, the two-dimensional spectra of excitation and emission were calibrated. The number of excitations per unit time in a membrane area Aₘₑₘ with n_{dye} molecules per unit area is Aₘₑₘn_{dye}∈(λₑₓ)I_{λ}^{ill}(λₑₓ)Δλₑₓ with the molecular cross section of absorption ∈(λₑₓ) and the quantum intensity of illumination per wavelength interval I_{λ}^{ill}(λₑₓ) at a bandwidth Δλₑₓ. The number of detected quanta per unit time P(λₑₓ,λₑₘ) is given by Eq. 1 with the quantum yield Φₑₘ, the normalized quantum spectrum of fluorescence per wavelength interval f_{λ}(λₑₘ) and the efficiency T^{rec}(λₑₘ) and bandwidth Δλₑₘ of the recording system.

P(λₑₓ,λₑₘ)=Aₘₑₘn_{dye}I_{λ}^{ill}(λₑₓ)Δλₑₓ∈(λₑₓ)Φₑₘf_{λ}(λₑₘ)T^{rec}(λₑₘ)Δλₑₘ (1)

We obtain a two-dimensional fluorescence spectrum that is defined by molecular parameters when we divide Eq. 1 by the number of dye molecules Aₘₑₘn_{dye} and by the spectra and bandwidths of illumination I_{λ}^{ill}(λₑₓ)Δλₑₓ and recording T^{rec}(λₑₘ)Δλₑₘ. The resulting fluorescence spectrum of excitation and emission per unit wave number of emission F _{ν̅}( ν̅ₑₓ, ν̅ₑₘ)=∈( ν̅ₑₓ)Φₑₘf _{ν̅}( ν̅ₑₘ) is given by Eq. 2, considering the relations f _{ν̅}( ν̅ₑₘ)=λₑₘ²f_{λ}(λₑₘ) and ∈( ν̅ₑₓ)=∈(λₑₓ).

$${F}_{\overline{v}} ( {\overline{v}}_{ex} , {\overline{v}}_{em} ) = \frac{{λ}_{em}^{2}}{{A}_{mem} {n}_{dye} {I}_{λ}^{ill} ( {λ}_{ex} ) Δ {λ}_{ex} {T}^{rec} ( {λ}_{em} ) Δ {λ}_{em}} P ( {λ}_{ex} , {λ}_{em} ) \qquad ( 2 )$$

T^{rec}(λₑₘ)Δλₑₘ is obtained up to an arbitrary factor by calibrating the recording system (objective, dichroic mirror, long pass filter, 0.6 mm fibre optics, monochromator and diode array). We place a standard lamp (OL245M, Optronic Laboratories, Orlando, USA) with a known quantum spectrum I_{λ}^{cal}(λ) on the microscope and measure the response P^{cal}(λ)=I_{λ}^{cal}(λ)T^{rec}(λₑₘ)Δλₑₘ of the photodiode array. We probe the illumination (Xe lamp, monochromator, optical fibre, broadening optics, dichroic mirror and objective) with a calibrated detector of efficiency T^{cal}(λ) and bandwidth Δλ. For that purpose we use part of our recording system—the 0.6 nm optical fiber with monochromator and diode array—that is again calibrated by illuminating the fiber end with the standard lamp. With the fiber end on the microscope, we measure P^{ill}(λ)=I_{λ}^{ill}(λₑₓ^{nom},λ)T^{cal}(λ)Δλ for each λₑₓ^{nom} set at the monochromator. The resulting illumination spectra I_{λ}^{ill}(λₑₓ^{nom},λ) are fitted with Gaussians with a maximum defining the excitation wavelength λₑₓ and an integral that represents the intensity I_{λ}^{ill}(λₑₓ)Δλₑₓ up to a constant factor.

Protocol. The measurements were started 30 min after staining. Under voltage clamp, the voltage sensitive fluorescence is investigated with the following protocol: (1) opening of the shutter, (2) selection of an excitation wavelength λₑₓ, (3) after a delay of 100 ms hyperpolarization of the cell to V_{M}=−70 mV, (4) recording of a complete emission spectrum in a range of λₑₘ=510-833 nm for RH-421, di-4-ANEPBS, BNBIQ and ANNINE-6 and λₑₘ=460-833 nm for ANNINE-5 with an integration time of 200 ms, (5) after a delay of 100 ms depolarization to about V_{M}=10 mV, (6) recording of two complete emission spectra, (7) after a delay of 100 ms hyperpolarization to V_{M}=−70 mV, (8) recording of another complete emission spectrum, (9) closing of the shutter. The steps (2)-(8) are repeated for various wavelengths of excitation in the range λₑₓ=360-510 nm for RH-421, di-4-ANEPBS, BNBIQ and ANNINE-6 and λₑₓ=360-460 nm for ANNINE-5 at a stepwidth of 5 nm. At each excitation wavelength the two spectra of the hyperpolarized cell are averaged as well as the two spectra of the depolarized cell to obtain the spectra P(λₑₓ,λₑₘ) at the two voltages. The two-dimensional fluorescence spectra F _{ν̅}( ν̅ₑₓ, ν̅ₑₘ) are computed according to Eq. 2 up to a constant factor using the spectra I_{λ}^{ill}(λₑₓ)Δλₑₓ and T^{rec}(λₑₘ)Δλₑₘ given by calibration. At the hyperpolarized voltage the spectrum is scaled to its maximum as F _{ν̅}^{hyp}/F _{ν̅}^{hyp,MAX}. At the depolarized voltage the spectrum is scaled by the same factor as F _{ν̅}^{dep}/F _{ν̅}^{hyp,MAX}.

2D spectra. The relative fluorescence spectra per wave number interval of emission F _{ν̅}( ν̅ₑₓ, ν̅ₑₘ)/F _{ν̅}^{MAX} Retzius neurons at a voltage V_{M}=−70 mV are plotted in the left column of FIG. 3 for RH-421, di4-ANEPBS, BNBIQ and ANNINE-5 and ANNINE-6. They are limited at the red and of excitation and the blue end of emission by the dichroic mirror of the microspectrometer. The maxima reflect the position of the upward and downward vibroelectronic transition between the S₀ and S₁ states of the dyes. They are shifted to higher wave numbers of excitation and emission in the homologous series RH-421, di4-ANEPBS, BNBIQ and ANNINE-5 and also in ANNINE-6. With respect to excitation, a second band appears in the ANNINEs at high wave numbers due to the S₀/S₂ transition.

1D spectra. In order to study the interdependence of excitation and emission, we consider emission spectra per wave number of emission F _{ν̅}( ν̅ₑₘ) at various wave numbers of excitation ν̅ₑₓ and excitation spectra F _{ν̅}( ν̅ₑₓ) per wave number of emission for various wave numbers of emission ν̅ₑₘ in the two-dimensional spectra. These one-dimensional spectra are fitted by log normal functions [27] according to Eq. 3 with an amplitude F _{ν̅}^{MAX}, a spectral maximum ν̅^{MAX}, a spectral width W and a spectral asymmetry b, excluding the contribution of the S₀/S₂ transition.

$$\frac{{F}_{\overline{v}} ( \overline{v} )}{{F}_{\overline{v}}^{MAX}} = exp \{ - \frac{{ln}^{2} [ 1 + 2 b ( \overline{v} - {\overline{v}}^{MAX} ) / W ]}{{b}^{2} / ln 2} \} \qquad ( 3 )$$

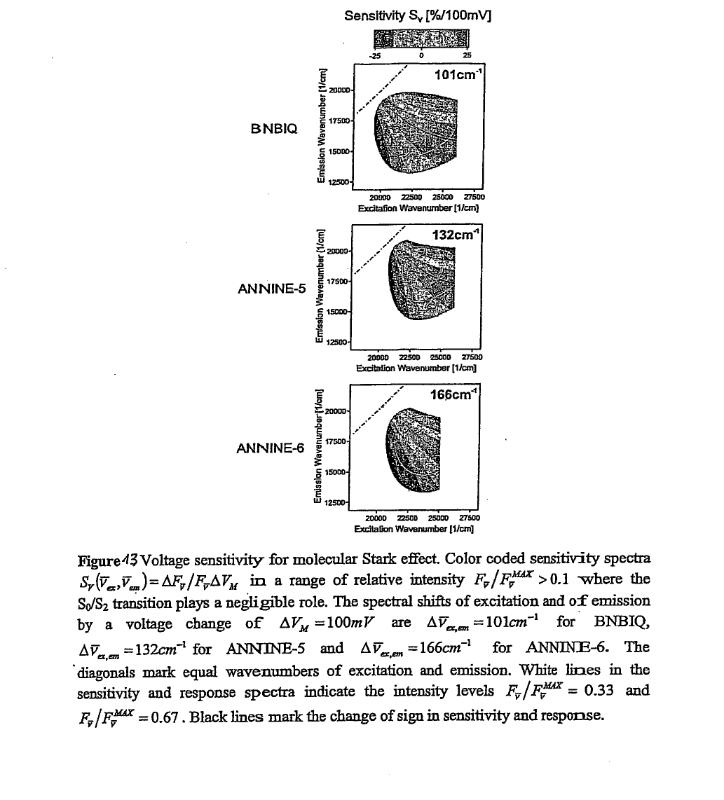

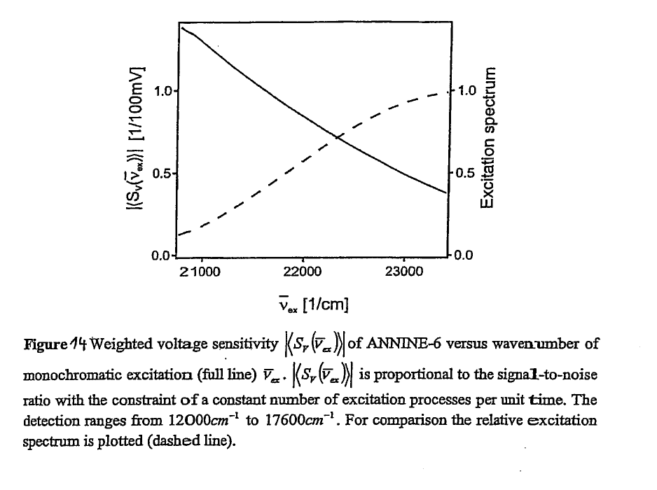

FIG. 9 shows the parameters of the emission spectra ν̅ₑₘ^{MAX}, Wₑₘ and bₑₘ as a function of the excitation wave number ν̅ₑₓ and the parameters of the excitation spectra ν̅ₑₓ^{MAX}, Wₑₓ and bₑₓ as a function of the emission wave number ν̅ₑₘ. The maximum of excitation is shifted to the blue at high wave numbers of emission, and the maximum of emission is shifted to the blue for higher wave numbers of excitation. Such effects are expected for hemicyanine dyes when the solvent shell is incompletely relaxed in the excited state [16]. However, these shifts of the spectral maxima as well as all other changes of spectral width and spectral asymmetry displayed in FIG. 9 are rather small compared with the width of the spectra and the difference of excitation and emission maxima. Considering these observations with the one-dimensional spectra, we use in the following evaluation of the two-dimensional spectra the approximation that excitation and emission processes are independent of each other.

Fit of 2D spectra. In order to attain a well defined parametrization of the fluorescence spectra, we fit the normalized 2D spectra F _{ν̅}( ν̅ₑₓ, ν̅ₑₘ)/F _{ν̅}^{MAX} shown in the left column of FIG. 3 by products of two log normal spectra according to Eq. 4.

$$\frac{{F}_{\overline{v}} ( {\overline{v}}_{ex} , {\overline{v}}_{em} )}{{F}_{\overline{v}}^{MAX}} = exp \{ - \frac{{ln}^{2} [ 1 + 2 {b}_{ex} ( \overline{v} - {\overline{v}}_{ex}^{MAX} ) / {W}_{ex} ]}{{b}_{ex}^{2} / ln 2} \} exp \{ - \frac{{ln}^{2} [ 1 + 2 {b}_{em} ( \overline{v} - {\overline{v}}_{em}^{MAX} ) / {W}_{em} ]}{{b}_{em}^{2} / ln 2} \} \qquad ( 4 )$$

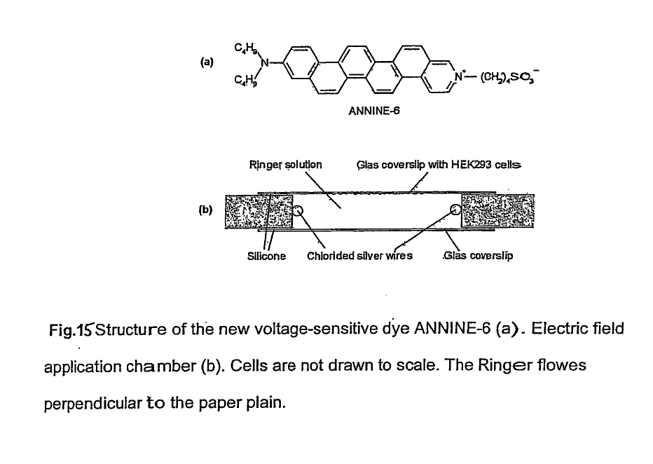

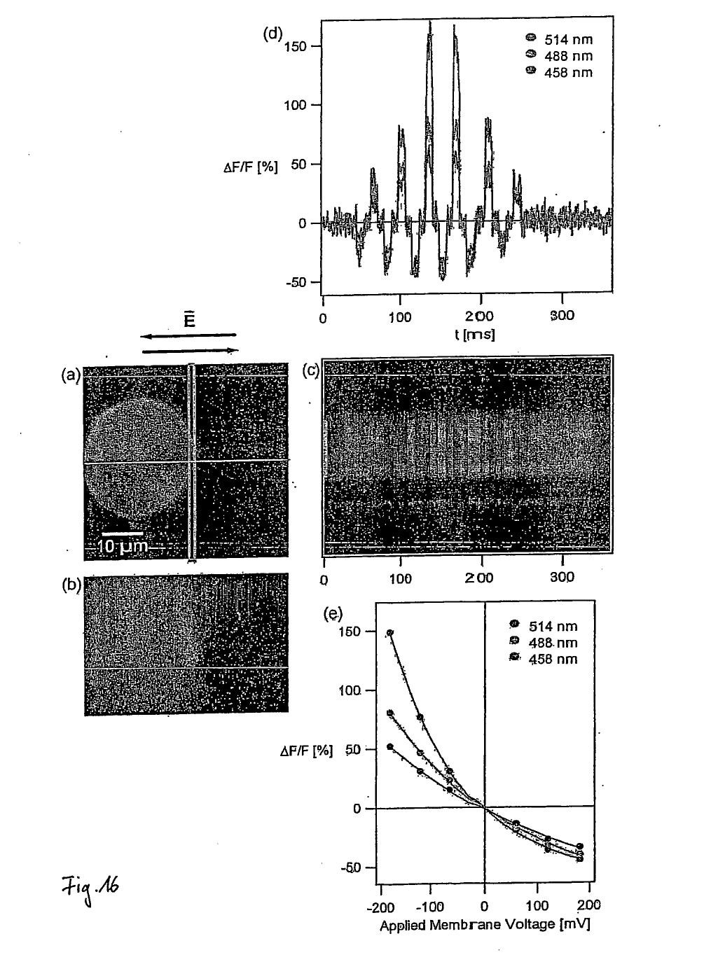

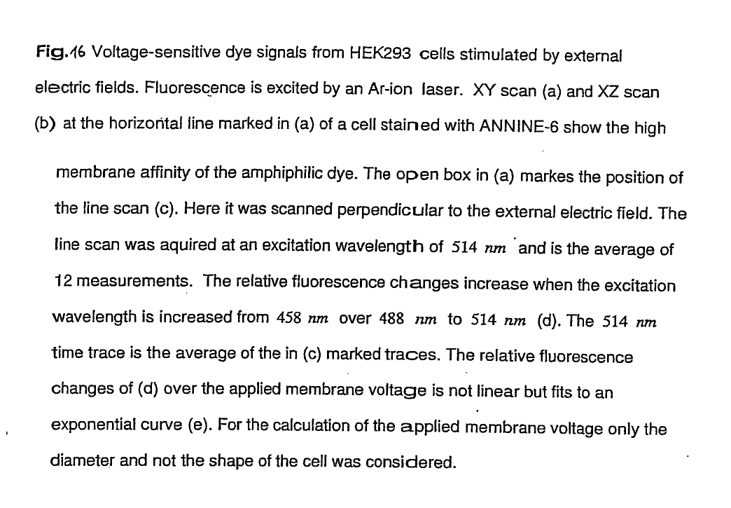

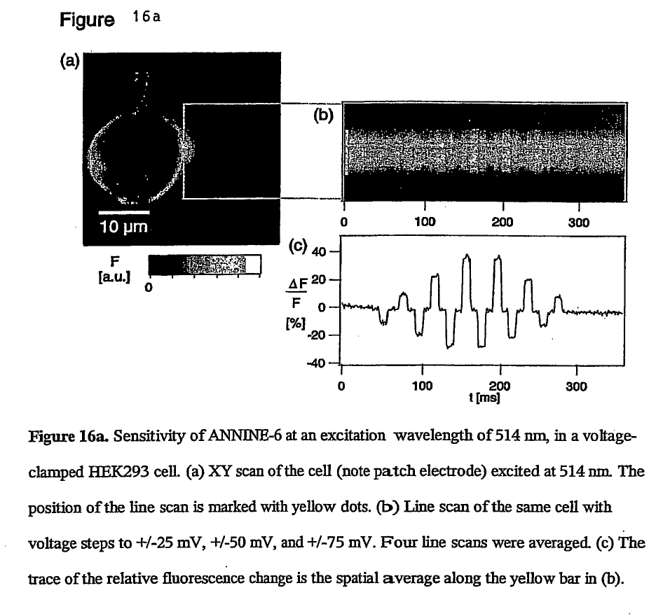

Considering F _{ν̅}( ν̅ₑₓ, ν̅ₑₘ)=∈( ν̅ₑₓ)Φₑₘf _{ν̅}( ν̅ₑₘ) with the cross section of absorption ∈( ν̅ₑₓ) and the quantum yield Φₑₘ and normalized quantum spectrum of emission f _{ν̅}( ν̅ₑₘ), the product function of Eq. 4 accounts for the spectral shape of absorption and emission. The five sets of the six spectral parameters are summarized in Table 1, the fitted 2D spectra are displayed in the left column of FIG. 10. We note a distinct asymmetry of the 2D spectra, in particular for ANNINE-5 and ANNINE-6 with steep slopes towards the red in excitation and towards the blue in emission. To illustrate the quality of the fit, we check one-dimensional sections along the axes of excitation and emission. The upper graphs of FIG. 11 show the data and the fit function. The good agreement—except for high wave numbers of excitation where the contribution of the S₀/S₂ transition is significant—confirms the validity of the approach. Also the one-dimensional spectra exhibit the steep slopes in the red of the excitation band and in the blue of the emission band.

Voltage sensitivity. The spectrum of voltage sensitivity S_{V}( ν̅ₑₓ, ν̅ₑₘ) is defined as the relative change of fluorescence intensity per change of membrane voltage according to Eq. 5.

$${S}_{V} ( {\overline{v}}_{ex} , {\overline{v}}_{em} ) = \frac{Δ {F}_{\overline{v}} ( {\overline{v}}_{ex} , {\overline{v}}_{em} )}{{F}_{\overline{v}} ( {\overline{v}}_{ex} , {\overline{v}}_{em} )} \frac{1}{Δ {V}_{M}} \qquad ( 5 )$$

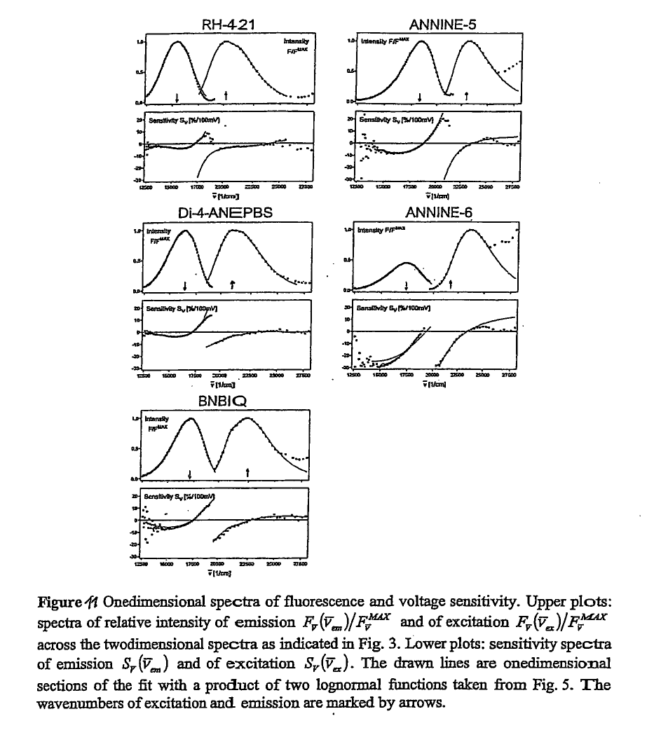

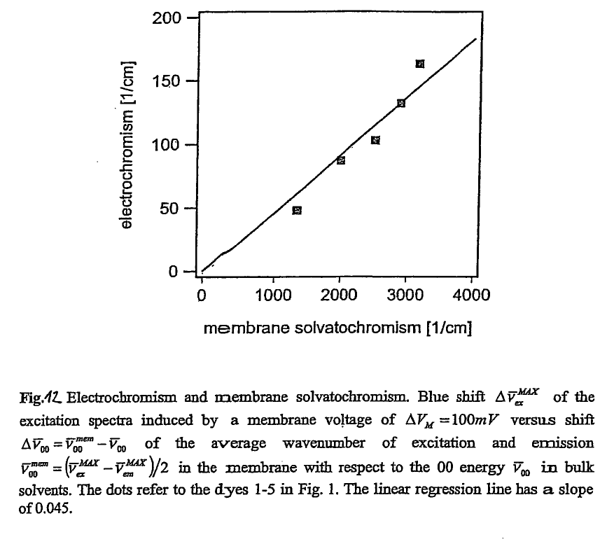

We obtain it by subtracting the relative spectrum F _{ν̅}^{hyp}/F _{ν̅}^{hyp,MAX} at a hyperpolarized voltage from the scaled spectrum F _{ν̅}^{dep}/F _{ν̅}^{hyp,MAX} at a depolarized voltage and dividing by F _{ν̅}^{hyp}/F _{ν̅}^{hyp,MAX} and the voltage difference ΔV_{M}. The results—scaled to a voltage ΔV_{M}=100 mV—are shown in the right column of FIG. 8. All sensitivity spectra exhibit two regions: at high wave numbers of excitation and emission, the fluorescence is enhanced, at low wave numbers the fluorescence is reduced. Tha magnitude of this effect increases in the homologous series RH-421, di4-ANEPBS, BNBIQ and ANNINE-5, and also in ANNINE-6. Within the limited spectral window of the measurements, the sensitivity is in the range of ±100%/100 mV for RH-421, ±15%/100 mV for di-4-ANEPPS, ±20%/100 mV for BNBIQ and ±25%/100 mV for ANNINE-5 and ANNINE-6.

Parametrized voltage sensitivity. A parametrization of voltage sensitivity is achieved by fitting the scaled spectra F _{ν̅}^{dep}/F _{ν̅}^{hyp,MAX} and F _{ν̅}^{hyp}/F _{ν̅}^{hyp,MAX} at the two voltages by products of log normal functions according to Eq. 4. The resulting differences of the spectral parameters Δ ν̅ₑₓ^{MAX}, Δ ν̅ₑₘ^{MAX}, ΔWₑₓ, ΔWₑₘ, Δbₑₓ, Δbₑₘ and ΔF _{ν̅}^{MAX} are small for all dyes. For that reason we can assume that the changes of the spectra are linear with respect to changes of the spectral parameters, and we are allowed to scale the parameter changes to a standard voltage change of ΔV_{M}=100 mV as it is typical for neuronal excitation. The resulting differences are shown in Table 2.

Using the fit parameters from Tables 1 and 2, we reconstruct the sensitivity spectra S_{V}( ν̅ₑₓ, ν̅ₑₘ) according to Eq. 5. The results are displayed in the central column of FIG. 10 in a range where the relative change of intensity is above 10%. For ANNINE-6, ANNINE-5 and BNBIQ, the fitted sensitivity spectra exhibit a distinct enhancement and reduction of fluorescence at high and low wave numbers, respectively. In particular, we note the steep increase of negative sensitivity with decreasing wave number of excitation in the red corner of the 2D spectrum and the steep increase of positive sensitivity with increasing wave number of emission in the blue corner of the 2D spectrum. For the two styryl dyes the sensitivity spectra are more involved.

To illustrate the quality of the sensitivity spectra S_{V}( ν̅ₑₓ, ν̅ₑₘ) reconstructed from the fit of the two-dimensional spectra at two voltages, we select one-dimensional spectra S_{V}( ν̅ₑₓ) and S_{V}( ν̅ₑₘ) across the sensitivity data (FIG. 8) and across the fitted spectra (FIG. 10). They are shown in FIG. 11 and exhibit a good agreement, except for high excitation wave numbers where the S₀/S₂ transition contributes and at low emission wave numbers where the intensity is modest.

Voltage sensitivity. An electric field across a cell membrane may in principle affect the electronic structure of a bound chromophore, its vibroelectronic coupling and its interaction with the matrix. It may change the electronic 00 transition energy, the Franck-Condon factors of excitation and emission, the transition dipole moments and radiationless deactivation channels. As a result, the two-dimensional fluorescence spectrum F _{ν̅}( ν̅ₑₓ, ν̅ₑₘ)=∈( ν̅ₑₓ)Φₑₘf _{ν̅}( ν̅ₑₘ) may be modulated through the absorption spectrum ∈( ν̅ₑₓ), the quantum yield of emission Φₑₘ and the normalized quantum spectrum of emission f _{ν̅}( ν̅ₑₘ). The sensitivity spectrum defined by Eq. 5 reflects changes of all these molecular parameters according to Eq. 6.

$${S}_{V} ( {\overline{v}}_{ex} , {\overline{v}}_{em} ) = \frac{1}{ɛ ( {\overline{v}}_{ex} )} \frac{Δɛ ( {\overline{v}}_{ex} )}{Δ {V}_{M}} + \frac{1}{{Φ}_{em}} \frac{Δ {Φ}_{em}}{Δ {V}_{M}} + \frac{1}{{f}_{\overline{v}} ( {\overline{v}}_{em} )} \frac{Δ {f}_{\overline{v}} ( {\overline{v}}_{em} )}{Δ {V}_{M}} \qquad ( 6 )$$

In fact, when we fit the two-dimensional fluorescence spectra at two voltages by products of log normal functions, we find that the joint amplitude F _{ν̅}^{MAX} and all spectral parameters of excitation ν̅ₑₓ^{MAX}, Wₑₓ, bₑₓ and of emission ν̅ₑₘ^{MAX}, Wₑₘ, bₑₘ are modified by an electric field as summarized in Table 2. An assignment of these parameter changes to molecular mechanisms, however, is difficult. E.g. spectral shifts Δ ν̅ₑₓ^{MAX} and Δ ν̅ₑₘ^{MAX} may arise not only from a change of the electronic 00 energy, and amplitude changes ΔF _{ν̅}^{MAX} not only from a changed fluorescence quantum yield, but in both cases changes of spectral shape contribute, too. It is a further problem that the parameter changes Δ ν̅ₑₓ^{MAX}, Δ ν̅ₑₘ^{MAX}, ΔWₑₓ, ΔWₑₘ, Δbₑₓ, Δbₑₘ and ΔF _{ν̅}^{MAX} are derived from a small difference of two spectra, a procedure that implies a large error. For both reasons we do not attempt to provide a detailed mechanistic interpretation of voltage-sensitivity. We consider only one aspect, the possible contribution of a molecular Stark effect.

Electrochromism of excitation. A typical feature of voltage sensitivity of all hemicyanines considered is a blue shift Δ ν̅ₑₓ^{MAX} of all excitation spectra induced by a positive change ΔV_{M} of the membrane voltage (Table 2). That blue shift increases in the homologous series RH-421, di-4-ANEPBS, BNBIQ, ANNINE-5 and also in ANNINE-6. To confirm the significance of that observation, we repeat the evaluation of the spectral data in terms of a product of log normal functions (Eq. 4) with five parameters at constant spectral asymmetries bₑₓ and bₑₘ and also with three parameters at constant asymmetries and constant spectral widths Wₑₓ and Wₑₘ. The resulting spectral shifts are summarized in Table 3. Apparently, the systematic blue shift of excitation is rather insensitive to the fit procedure.

We assign the blue shift of excitation to a molecular Stark effect of the membrane bound chromophores [28-31]. Due to their amphiphilic structure, the hemicyanines may be expected to be bound to the surface of the neuron membrane with a distinct orientation [18,19]. In that case, an intramolecular charge displacement Δμ_{EG} by electronic excitation from the pyridinium to the aniline moiety is directed against a change ΔE=ΔV_{M}/d_{M} of the average electrical field in a membrane of thickness d_{M} as induced by a positive change ΔV_{M} of the membrane voltage. The expected blue shift is expressed by Eq. 7 with a projection cos θ of charge displacement on the membrane normal (Planck's constant h, velocity of light c).

hcΔ ν̅ₑₓ^{MAX}=Δμ_{EG}ΔE cos θ (7)

From the experimental shifts Δ ν̅ₑₓ^{MAX} we estimate the charge displacement Δμ_{BG} cos θ along the membrane normal for ΔV_{M}=100 mV and d_{M}=4 nm. Considering the fit with seven parameters (Table 2), we obtain for RH-421, di-4-ANEPBS, BNBIQ, ANNINE-5 and ANNINE-6 values of 12, 21, 24, 31 and 39 Debye. The effective displacements of an elementary charge e₀ along the membrane normal (Δμ_{EG}/e₀) cos θ are 0.24 nm, 0.43 nm, 0.51 nm, 0.65 nm and 0.81 nm. With respect to charge displacement, ANNINE-6 is better by factor of three than the classical styryl dye RH-421. The enhancement may be due either to a stronger intramolecular charge shift Δμ_{EG} or to a better orientation cos θ in the membrane. A discrimination of the two effects is not possible on the basis of the present data. It may be achieved by systematic measurements of the chromophore orientation in the neuron membrane and by quantum chemical computations of the electronic structure of the chromophores.

Electrochromism and membrane solvatochromism. The assignment of a Stark effect to the electrochromic blue shift is confirmed by a consideration of solvatochromism in the neuron membrane. Due to their orientation, the chromophores at the membrane/water interface are in an extremely inhomogeneous environment that may give rise to specific solvatochromic effects with amphiphilic hemicyanines [33]. We have to distinguish the effect of local polarity and the effect of the local polarity gradient [33]. In homogeneous solvents, a changing polarity gives rise to a symmetric opposite shift of excitation and emission spectra with an invariant average wave number ν̅₀₀=( ν̅ₑₓ^{MAX}+ ν̅ₑₘ^{MAX})/2 of the 00 energy [12]. As a consequence, a shift Δ ν̅₀₀= ν̅₀₀^{mem}− ν̅₀₀ of the average wave number in the membrane may be considered as a quantitative indicator for an effect of the polarity gradient. The wave numbers ν̅₀₀^{mem} in the neuron membrane and the 00 energies ν̅₀₀ in bulk solvents [12,23] are summarized in Table 4. There is indeed a large blue shift for all dyes that increases in the series RH-421, di-4-ANEPBS, BNBIQ, ANNINE-5 and ANNINE-6.

We may expect that the solvatochromic effect of the polarity gradient is related to the intramolecular charge displacement Δμ_{EG} cos θ across the polarity gradient. Thus we express the blue shift Δ ν̅₀₀ by Eq. 8

hcΔ ν̅₀₀=E₀Δμ_{EG} cos θ (8)

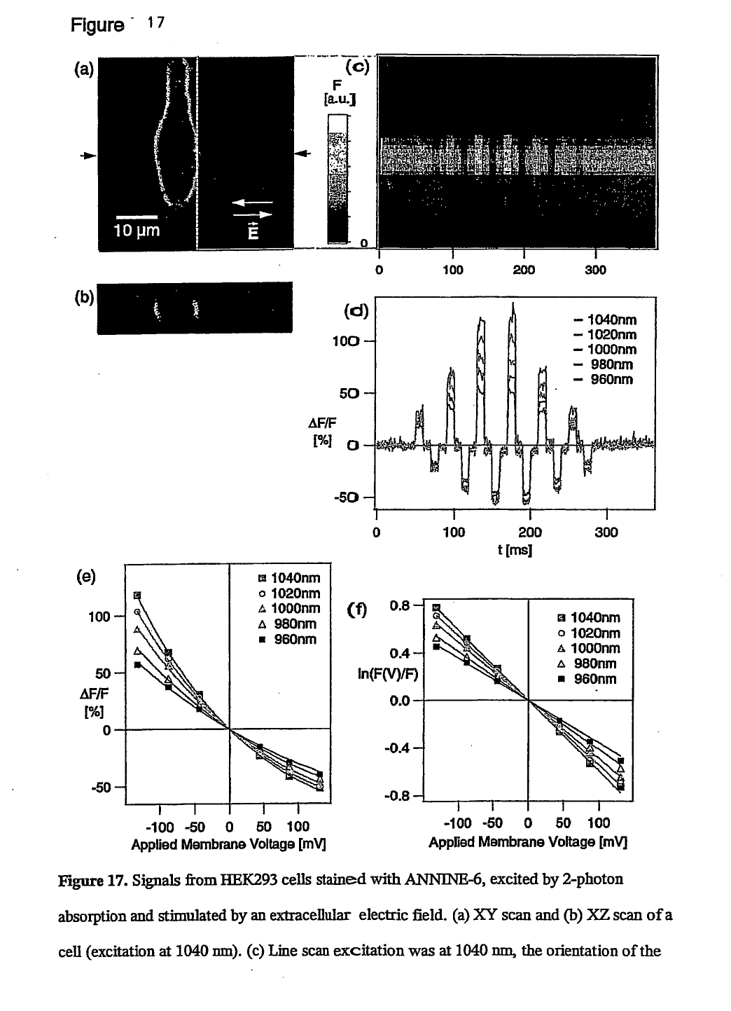

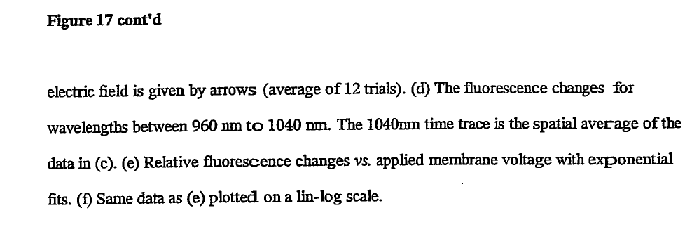

The effective internal electric field E₀ characterizes the strength of the gradient. On the basis of Eq. 8 the increasing solvatochromic blue shifts Δ ν̅₀₀ in the series RH-421, di-4-ANEPBS, BNBIQ, ANNINE-5 and ANNINE-6 reflect an increasing charge displacement Δμ_{EG} cos θ. As electrochromism caused by a Stark effect and solvatochromism induced by a polarity gradient are both proportional to Δμ_{EG} cos θ, we expect a proportionality of the electrochromic shift Δ ν̅ₑₓ^{MAX} and the solvatochromic shift Δ ν̅₀₀. In fact, FIG. 12 shows such a linear correlation with Δ ν̅ₑₓ^{MAX}=0.045Δ ν̅₀₀ and confirms the consistency of our interpretations.

Ideal Stark effect probes. A molecular Stark effect may affect not only the excitation but also the emission of a dye. The resulting spectral shift is described by Eq. 9.

hcΔ ν̅ₑₘ^{MAX}=Δμ_{EG}ΔE cos θ (9)

In fact, for BNBIQ, ANNINE-5 and ANNINE-6 we observe electrochromic blue shifts of emission that are almost identical to those of excitation (Table 3). We assign that correspondence to an identical molecular Stark effect for excitation and emission with an identical charge shift Δμ_{EG} cos θ due to an identical electronic transition and an similar orientation of the chromophore for excitation and emission.

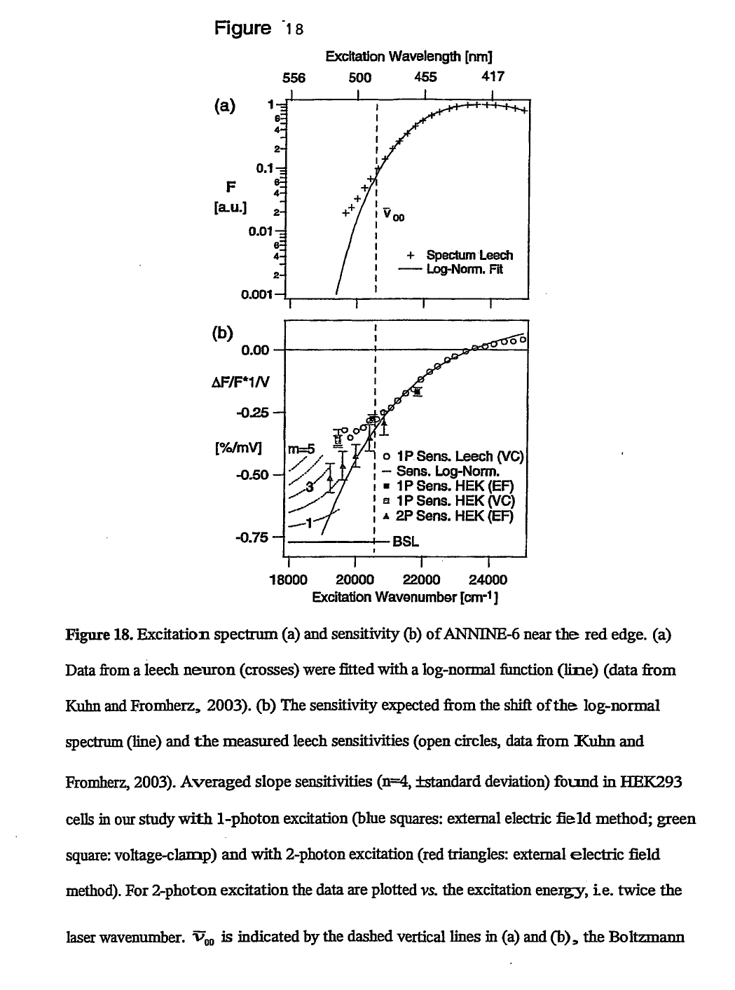

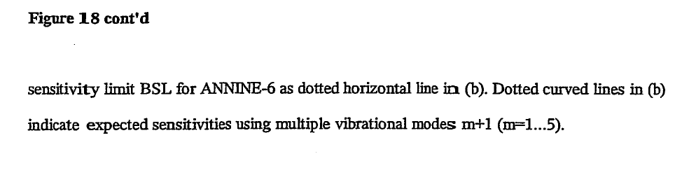

To test, how far the Stark effect of excitation and emission dominates the voltage sensitivity of BNBIQ, ANNINE-5 and ANNINE-6, we compute the sensitivity spectrum S_{V}( ν̅ₑₓ, ν̅ₑₘ) for an identical blue shift of excitation and emission at invariant spectral shape and amplitude with ΔWₑₓ=ΔWₑₘ=Δbₑₓ=Δbₑₘ=ΔF _{ν̅}^{MAX}=0 using Eqs. 4 and 5. Choosing the averages Δ ν̅_{ex,em}^{MAX}=(Δ ν̅ₑₓ^{MAX}+Δ ν̅ₑₘ^{MAX})/2 of the blue shifts from Table 2, we obtain FIG. 13. The computed voltage sensitivities are most similar to the fitted spectra in the central column of FIG. 10: BNBIQ, ANNINE-5 and ANNINE-6 are almost ideal Stark probes for voltage changes in nerve cells.

When we express the voltage sensitivity for a pure Stark effect by Eq. 6, considering a small spectral shift Δ ν̅_{ex,em}, we obtain Eq. 10 in terms of the derivatives of the absorption and emission spectrum ∈′=d∈/d ν̅ₑₓ and f′=df/d ν̅ₑₘ.

$${S}_{V} ( {\overline{v}}_{ex} , {\overline{v}}_{em} ) = \frac{Δ {\overline{v}}_{ex , em}}{Δ {V}_{M}} [ \frac{{ɛ}^{′} ( {\overline{v}}_{ex} )}{ɛ ( {\overline{v}}_{ex} )} + \frac{{f}_{\overline{v}}^{′} ( {\overline{v}}_{em} )}{{f}_{\overline{v}} ( {\overline{v}}_{em} )} ] \qquad ( 10 )$$

The voltage sensitivity S_{V}( ν̅ₑₓ, ν̅ₑₘ) depends not only on the spectral shift caused by the intramolecular charge displacement, but also on the shapes of the absorption and emission spectra that are determined by the Franck-Condon factors of the vibroelectronic transitions. The relative slope of the excitation spectrum for ANNINE-5 and ANNINE-6 is highest and positive in the red, whereas the relative slope of the emission spectrum is highest and negative in the blue as shown in FIGS. 10 and 11. The relative slopes are distinctly smaller in the blue of excitation and in the red of emission. However, due to their different sign, the optimal sensitivities with respect to excitation and emission cannot be combined. For optical recording we may choose the red corner of the two-dimensional sensitivity spectra as illustrated in FIG. 10. In accordance with Eq. 10, the sensitivity increases dramatically in the red of excitation but only modestly in the red of emission as illustrated also by the one-dimensional spectra of FIG. 11.

Styryl dyes. For the styryl dyes di-4-ANEPBS and RH421 the spectral shifts of excitation and emission are not identical (Table 2). The blue shift of emission for di-4-ANEPBS is about 50% of the blue shift of excitation, and RH-421 even exhibits a red shift of emission. These results do not depend on the fit procedure (Table 3). Apparently, with these dyes the emission is affected by other effects besides a Stark effect. Possibly, the electrical field changes position and orientation of the excited chromophores, resulting in a field induced solvatochromic red shift of emission that is superimposed to a Stark effect.

Optical recording. The sensitivity function S_{V}( ν̅ₑₓ, ν̅ₑₘ) reflects molecular features of the membrane bound dye according to Eq. 6. However, the photodiode signal ΔP( ν̅ₑₓ, ν̅ₑₘ) due to a voltage change ΔV_{M} of neuronal activity in an experimental setup of optical recording does not only depend on voltage sensitivity, but also on staining, illumination and detection. Considering Eqs. 1 and 2 we obtain Eq. 11: a high response requires a large observed membrane area Aₘₑₘ, a high density of dye molecules per unit area n_{dye}, a high quantum intensity of illumination I _{ν̅}^{ill}( ν̅ₑₓ)Δ ν̅ₑₓ, a high efficiency of the detection system T^{rec}( ν̅ₑₘ)Δ ν̅ₑₘ, a high voltage sensitivity S_{V}( ν̅ₑₓ ν̅ₑₘ) and—considering F _{ν̅}( ν̅ₑₓ, ν̅ₑₘ)=∈( ν̅ₑₓ)f _{ν̅}( ν̅ₑₘ)Φₑₘ—a high cross section of absorption ∈( ν̅ₑₓ) and a high yield of fluorescence f _{ν̅}( ν̅ₑₘ)Φₑₘ.

ΔP( ν̅ₑₓ, ν̅ₑₘ)=ΔV_{M}Aₘₑₘn_{dye}·I _{ν̅}^{ill}( ν̅ₑₓ)Δ ν̅ₑₓ·S_{V}( ν̅ₑₓ, ν̅ₑₘ)·F _{ν̅}( ν̅ₑₓ, ν̅ₑₘ)·T^{rec}( ν̅ₑₘ)Δ ν̅ₑₘ (11)

We consider an illumination with a narrow spectral band width at a wave number ν̅*ₑₓ with an integral intensity I^{ill}=∫I _{ν̅}^{ill}( ν̅ₑₓ)d ν̅ₑₓ and a detection with constant efficiency T^{rec}( ν̅ₑₘ)=T^{rec} within the spectral limits ν̅ₑₘ^{A} and ν̅ₑₘ^{B} of recording. The total photodiode signal is given by Eq. 12.

$$Δ P = Δ {V}_{M} {A}_{mem} {n}_{dye} {I}^{ill} {T}^{rec} {\int }_{\overline{v} emA}^{\overline{v} emB} {F}_{\overline{v}} ( {\overline{v}}_{ex}^{*} , {\overline{v}}_{em} ) {S}_{V} ( {\overline{v}}_{ex}^{*} , {\overline{v}}_{em} ) d {\overline{v}}_{em} \qquad ( 12 )$$

An optimal signal is achieved by good staining, high illumination intensity, high detector sensitivity and proper selection of the excitation wave number ν̅*ₑₓ and the emission filters ν̅ₑₘ^{A} and ν̅ₑₘ^{B} to attain a large integral over the product of sensitivity spectrum and fluorescence spectrum. For illustration the normalized response spectrum R_{V}( ν̅ₑₓ, ν̅ₑₘ)=S_{V}( ν̅ₑₓ, ν̅ₑₘ)F _{ν̅}( ν̅ₑₓ, ν̅ₑₘ)/F _{ν̅}^{MAX} is plotted in the right column of FIG. 10. Highest negative integrals are found in the red corner of the spectrum with an excitation wave number ν̅*ₑₓ at the minimum of R_{V}( ν̅ₑₓ, ν̅ₑₘ) and with emission filters ν̅ₑₘ^{A} and ν̅ₑₘ^{B} excluding the region of positive response. The highest response increases significantly in the series RH-421, di-4-ANEPBS, BNBIQ, ANNINE-5 and ANNINE-6.

Signal-to-noise. The total response expressed by Eq. 12 reflects the signal-to-noise ratio only if the noise is constant, e.g. dominated by the electronic amplifier. If the shot noise of photons dominates [34], we have to consider a noise level N=\sqrt{P/2τ_{D}} that depends on the total photon counts P and the time constant of the detector τ_{D}. With

$$P = {A}_{mem} {n}_{dye} {I}^{ill} {T}^{rec} {\int }_{\overline{v} em A}^{\overline{v} emB} {F}_{\overline{v}} ( {\overline{v}}_{ex}^{*} , {\overline{v}}_{em} ) d {\overline{v}}_{em}$$

we obtain Eq. 13.

$$\frac{Δ P}{N} = Δ {V}_{M} \sqrt{2 {τ}_{D} {A}_{mem} {I}^{ill} {n}_{dye} {T}^{rec} {Φ}_{em}} \\ \frac{{\int }_{Tem A}^{Tem B} {F}_{\overline{v}} ( {\overline{v}}_{ex}^{*} , {\overline{v}}_{em} ) {S}_{\overline{v}} ( {\overline{v}}_{ex}^{*} , {\overline{v}}_{em} ) d {\overline{v}}_{em}}{\sqrt{{\int }_{\overline{v} em A}^{\overline{v} em B} {F}_{\overline{v}} ( {\overline{v}}_{ex}^{*} , {\overline{v}}_{em} ) d {\overline{v}}_{em}}} \qquad ( 13 )$$

In optimizing the signal-to-noise ratio, we have to take into account the role of photobleaching and phototoxicity of the dyes. These effects impair optical recording because dyes and cells fade away by illumination. Both photochemical processes are determined by the number of excitations per unit time. The signal-to-noise ratio must be optimized with the constraint of a certain number of excitations per unit time that is tolerable for a certain experiment. When we express the fluorescence spectrum by F _{ν̅}( ν̅*ₑₓ, ν̅ₑₘ)=∈( ν̅*ₑₓ)Φₑₘf _{ν̅}( ν̅ₑₘ) we obtain Eq. 14.

$$\frac{Δ P}{N} = Δ {V}_{M} \sqrt{2 {τ}_{D} {A}_{mem}} \sqrt{{I}^{ill} {n}_{dye} ɛ ( {\overline{v}}_{ex}^{*} )} \sqrt{{T}^{rec} {Φ}_{em}} \\ \frac{{\int }_{Tem A}^{Tem B} {f}_{\overline{v}} ( {\overline{v}}_{em}^{*} ) {S}_{\overline{v}} ( {\overline{v}}_{ex}^{*} , {\overline{v}}_{em} ) d {\overline{v}}_{em}}{\sqrt{{\int }_{\overline{v} em A}^{\overline{v} em B} {f}_{\overline{v}} ( {\overline{v}}_{em} ) d {\overline{v}}_{em}}} \qquad ( 14 )$$

Here the signal-to-noise ratio is expressed in terms of (i) the spatiotemporal resolution τ_{D}Aₘₑₘ of the setup, (ii) the number of excitations per area and time I^{ill}n_{dye}∈( ν̅*ₑₓ), (iii) the yield of detected quanta T^{rec}Φₑₘ and (iv) the sensitivity function S _{ν̅}( ν̅*ₑₓ, ν̅ₑₘ) weighted by the fluorescence spectrum f _{ν̅}( ν̅ₑₘ) within the spectral limits of detection. With a constraint I^{ill}∈( ν̅*ₑₓ)=const. the choice of an optimal wave number of excitation ν̅*ₑₓ and of optimal emission filters ν̅ₑₘ^{A} and ν̅ₑₘ^{B} is determined by a weighted sensitivity function [symbol] S_{V}( ν̅*ₑₓ) [symbol] defined by Eq. 15.

$${S}_{V} ( {\overline{v}}_{ex}^{*} ) = \frac{{\int }_{\overline{v} emA}^{\overline{v} emB} {f}_{\overline{v}} ( {\overline{v}}_{em} ) {S}_{\overline{v}} ( {\overline{v}}_{ex}^{*} , {\overline{v}}_{em} ) d {\overline{v}}_{em}}{\sqrt{{\int }_{\overline{v} emA}^{\overline{v} emB} {f}_{\overline{v}} ( {\overline{v}}_{em} ) d {\overline{v}}_{em}}} \qquad ( 15 )$$

For the ANNINE dyes, the sensitivity S _{ν̅}( ν̅*ₑₓ, ν̅ₑₘ) (FIG. 10) reaches largest negative values in the red corner of the spectrum. We expect a high signal-to-noise ratio when we excite monochromatically at a rather low wave number ν̅*ₑₓ. To keep I^{ill}∈( ν̅*ₑₓ) constant, we have to compensate the low cross section of absorption by an enhanced illumination intensity. Of course, we have to limit the detection to the range of negative response. For illustration, the weighted sensitivity function [symbol] S_{V}( ν̅*ₑₓ) [symbol] obtained from the data is plotted in FIG. 14 for ANNINE-6 versus the wave number of illumination.

These features of the signal-to-noise ratio for I^{ill}∈( ν̅*ₑₓ)=const. are expected for a Stark effect. When we insert Eq. 10 in Eq. 15 we obtain Eq. 16.

$${S}_{V} ( {\overline{v}}_{ex}^{*} ) = \frac{Δ {\overline{v}}_{ex , em}}{Δ {V}_{M}} \sqrt{{\int }_{\overline{v} emA}^{\overline{v} emB} {f}_{\overline{v}} ( {\overline{v}}_{em} ) d {\overline{v}}_{em}} \\ \{ \frac{{ɛ}^{′} ( {\overline{v}}_{ex}^{*} )}{ɛ ( {v}_{ex}^{*} )} + \frac{{\int }_{\overline{v} emA}^{\overline{v} emB} {f}_{\overline{v}}^{′} ( {\overline{v}}_{em} ) d {\overline{v}}_{em}}{{\int }_{\overline{v} emA}^{\overline{v} emB} {f}_{\overline{v}} ( {\overline{v}}_{em} ) d {\overline{v}}_{em}} \} \qquad ( 16 )$$

When we choose an excitation wave number in the red where the relative slope of the excitation spectrum is steep, we expect an increasing contribution of the Stark effect of excitation at a rather invariant contribution of the Stark effect of emission. Thus we expect an increasing signal-to-noise ratio for an illumination ν̅*ₑₓ at the red end of the excitation spectrum. Of course, the signal-to-noise ratio is proportional to the spectral shift by the Stark effect.

The anellated hemicyanine dyes ANNINE-5 and ANNINE-6 exhibit voltage sensitivities in a neuron membrane that are distinctly higher than those of the classical styryl dyes. These novel probes rely on a well defined physical mechanism, the molecular Stark effect. Their excellence for optical recording of neuronal excitation is due to a large intramolecular charge shift in connection with suitable Franck-Condon factors of vibroelectronic transitions and a high quantum yield of fluorescence. Further improvements of the voltage sensitive dyes must be directed towards higher photochemical stability and lower phototoxicity and towards a selective staining of individual cells in a tissue.
(1) Bullen, A.; Saggau, P. Optical Recording from Individual Neurons in Culture. In: Modern Techniques in Neuroscience Research; 1 ed.; Johansson, H., Ed.; Springer-Verlag: Berlin, 1999; pp 89.
(2) Sinha, S. R.; Saggau, P. Optical Recording from Populations of Neurons in Brain Slices. In Modern Techniques in Neuroscience Research; 1 ed; Johansson, H., Ed.; Springer-Verlag: Berlin, 1999; pp 459.
(3) Grinvald, A.; Shoham, D.; Shmuel, A.; Glaser, D.; Vanzetta, I.; Shtoyerman, E.; Slovin, H.; Wijnbergen, C.; Hildesheim, R.; Arieli A. In-vivo Optical Imaging of Cortical Architecture and Dynamics. In Modern Techniques in Neuroscience Research; 1 ed.; Johansson, H., Ed.; Springer-Verlag: Berlin, 1999; pp 893.
(4) Tasaki, I.; Watanabe, A.; Sandlin, R.; Carnay, L. Proc. Natl. Acad. Sci. USA 1968, 61, 883.
(5) Cohen, L. B.; Salzberg, B. M.; Davila, H. V.; Ross, W. N.; Landowne, D.; Waggoner, A. S.; Wang, C. H. J. Membrane Biol. 1974, 19, 1.
(6) Cohen, L. B.; Salzberg, B. M. Rev. Physiol. Biochem. Pharmacol. 1978, 83, 35.
(7) Loew, L. M.; Bonneville, G. W.; Surow, J. Biochemistry 1978, 17, 4065.
(8) Loew, L. M.; Simpson, L. L. Biophys. J. 1981, 34, 353.
(9) Fluhler, E.; Burnham, V. G.; Loew, L. M. Biochemistry 1985, 24, 5749.
(10) Grinvald, A.; Hildesheim, R.; Farber, I. C.; Anglister, L. Biophys. J. 1982, 39, 301.
(11) Grinvald, A.; Fine, A.; Farber, I. C.; Hildesheim, R. Biophys. J. 1983, 42, 195.
(12) Fromherz, P. J. Phys. Chem. 1995, 99, 7188.
(13) Fromherz, P.; Lambacher, A. Biochim. Biophys. Acta 1991, 1068, 149.
(14) Fromherz, P.; Muller, C. O. Biochim. Biophys. Acta 1993, 1150, 111.
(15) Ephardt, H.; Fromherz, P. J. Phys. Chem. 1989, 93, 7717.
(16) Rocker, C.; Heilemann, A.; Fromherz, P. J. Phys. Chem. 1996, 100, 12172.
(17) Fromherz, P.; Rocker, C. Ber. Bunsen-Ges. Phys. Chem. 1994, 98, 128.
(18) Visser, N. V.; van Hoek, A.; Visser, A. J.; Frank, J.; Apell, H. J.; Clarke, R. J. Biochemistry 1995, 34, 11777.
(19) Lambacher, A.; Fromherz, P. J. Phys. Chem. B 2001, 105, 343.
(20) Gupta, R. K.; Salzberg, B. M.; Grinvald, A.; Cohen, L. B.; Kamino, K.; Lesher, S.; Boyle, M. B.; Waggoner, A. S.; Wang, C. H. J. Membrane Biol. 1981, 58, 123.
(21) Fromherz, P.; Dambacher, K. H.; Ephardt, H.; Lambacher, A.; Muller, C. O.; Neigl, R.; Schaden, H.; Schenk, O.; Vetter, T. Ber. Bunsen-Ges. Phys. Chem. 1991, 95, 1333.
(22) Ephardt, H.; Fromherz, P. J. Phys. Chem. 1993, 97, 4540.
(23) Huebener, G.; Lambacher, A.; Fromherz, P. J. Phys. Chem. B submitted 2003.
(24) Dietzel, I. D.; Drapeau, P.; Nicholls, J. G. J. Physiol. (Lond.) 1986, 372, 191.
(25) Meyer, E.; Muller, C. O.; Fromherz, P. Eur. J. Neurosci. 1997, 9, 778.
(26) Hamill, O. P.; Marty, A.; Neher, B.; Sakmann, B.; Sigworth, F. J. Pflugers Arch. Physiol. 1981, 391, 85.
(27) Siano, D. B.; Metzler, D. E. J. Chem. Phys. 1969, 51, 1856.
(28) Junge, W.; Witt, H. T. Z. Naturforsch. B 1968, B 23, 244.
(29) Bucher, H.; Wiegand, J.; Snavely, B. B.; Beck, K. H.; Kuhn, H. Chem. Phys. Lett. 1969, 3, 508.
(30) Reich, R.; Schmidt, S. Ber. Bunsen-Ges. Phys. Chem. 1972, 76, 589.
(31) Schmidt, S.; Reich, R. Ber. Bunsen-Ges. Phys. Chem. 1972, 76, 1202.
(32) Loew, L. M.; Simpson, L.; Hassner, A.; Alexanian, V. J. Am. Chem. Soc. 1979, 101, 5439.
(33) Ephardt, H.; Fromherz, P. J. Phys. Chem. 1991, 95, 6792.
(34) Knopfel, T.; Fromherz, P. Z. Naturforsch. C 1987, 42, 986.

### EXAMPLE 4

### Large Voltage-Sensitive Dye Signals Measured with One and Two-Photon Excitation

ANNINE-6 is a new, completely anellated hemicyanine dye. Two-dimensional sensitivity spectra suggest the use of the extreme red edge of the excitation spectrum to achieve large relative fluorescence changes in response to membrane voltage changes. Several biological preparations were used for testing. First, HEK293 cells were stained by bath application of the dye. Alternating external electric fields generated by applying voltages between two Ag/AgCl-wires in a closed chamber (1). To achieve the high light intensities, which are necessary to excite the dye at the very red edge of the excitation spectrum, we used laser scanning microscopes with Ar-Ion Laser for one-photon excitation (488 nm, 514 nm) and Ti:Sapphire Laser for two-photon excitation (976 nm). The fluorescence changes measured with 1 kHz line scans followed the applied voltage, which was alternated every 10 ms, without discernible delay. We found relative fluorescence increases of more that 60%/100 mV for hyperpolarizing membrane potential changes and decreases of more than 40%/100 mV for depolarizing membrane potential changes at 514 nm excitation wavelength. The asymmetry of the fluorescence change is expected given the spectral shape and a pure electrochromic spectral shift. We found changes of +40%/100 mV and −30%/100 mV both for one-photon excitation at 488 nm and two-photon excitation at 976 nm. Preliminary measurements from cultured hippocampal slices using a soluble variant of ANNINE-6 show fast fluorescence changes of up to 8% upon stimulation of the Schaffer collaterals in CA1.
(1) D. Gross, L. M. Loew and W. Webb. Biophys. J., 50: 339-348, 1986.

### EXAMPLE 5

### High Sensitivity of Stark-Shift Voltage-Sensing Dyes by One- or Two-Photon Excitation Near the Red Spectral Edge

Sensitivity spectra of Stark-shift voltage sensitive dyes (VSDs), such as ANNINE-6, suggest the use of the extreme red edges of the excitation spectrum to achieve large fractional fluorescence changes with membrane voltage. This was tested in cultured HEK293 cells. Cells were illuminated with light at the very red edge of the dye's excitation spectrum, where the absorption cross section is as much as 100 times smaller than at its peak. The small-signal fractional fluorescence changes were −0.17%/mV, −0.28%/mV, and −0.35%/mV for one-photon excitation at 458 nm, 488 nm, and 514 nm, respectively, and −0.29%/mV, −0.43%/mV, and −0.52%/mV for two-photon excitation at 960 nm, 1000 nm, and 1040 nm, respectively. For large voltage swings the fluorescence changes became nonlinear, reaching 50% and −28% for 100 mV hyper- and depolarization, respectively, at 514 nm and 70% and −40% at 1040 nm. Such fractional sensitivities are about 5 times larger than what is commonly found with other voltage-sensing dyes and approach the theoretical limit given by the spectral Boltzmann tail.

Voltage-sensitive dyes (VSDs) are widely used for probing cellular membrane potentials (Grinvald et al., 1988; London et al., 1987; Meyer et al., 1997; Spors and Grinvald, 2002), but for dyes fast enough to measure neural signals the fractional fluorescence changes are rather small, compared to, for example, Ca²⁺ indicators. To overcome shot noise and achieve a sufficient signal-to-noise ratio (SNR) high fluorescence intensities are, therefore, needed. This, in turn, leads to photodynamic damage and quick photobleaching. This problem becomes increasingly more serious as spatial and temporal resolutions need to be increased and has prevented the application of VSD recordings to problems such as spike time synchronization (Salinas and Sejnowski, 2001; Singer, 1999), where VSDs would otherwise be the preferred tool. The amount of fluorescence light that is necessary to achieve a particular SNR for a given membrane voltage decreases strongly as the relative fluorescence change per unit of voltage change increases. An increase in that sensitivity will, therefore, reduce photodamage substantially. The voltage sensitivity is a function of the intrinsic properties of the dye, such as the size of the spectral shift, and can be particularly sensitive to the choice of excitation wavelength with the maximal sensitivity at the spectral edge as has been realized early on and demonstrated for a limited wavelength range (Loew, 1982). In this paper we demonstrate that the sensitivity becomes even larger at extreme wavelengths and approaches the thermodynamic voltage-sensitivity limit. There we took advantage of the recent synthesis of a new class of VSDs (Hübener et al., 2003), which shows a spectral change that is entirely due to the molecular Stark effect (Kuhn and Fromherz, 2003). In addition, the charge shift between ground and excited state, on which the spectral shift directly depends, is nearly twice as large for ANNINE-6 compared to the classical styryl dye di-4-ANEPBS (Fluhler et al., 1985; Kuhn and Fromherz, 2003). In this paper we show that the dye's sensitivity can be increased beyond the already high values shown in the initial characterization (Kuhn and Fromherz, 2003) by using excitation wavelengths at the extreme long-wavelength edge of the absorption spectrum by both one- and two-photon excitation (Denk et al., 1990).

### Cell Culture

HEK293 cells (DSMZ GmbH, Braunschweig, Germany) were cultured using standard methods on glass coverslips (diameter 24 mm, Assistent, Glaswarenfabrik Karl Hecht KG, Sondheim, Germany). The coverslips were coated with fibronectin (No. 68885, Boehringer-Mannheim, Germany) in PBS (140 mM NaCl, 2.7 mM KCl, 1.5 mM KH₂PO₄, 6.5 mM Na₂HPO₄×2H₂O) by incubation for 45 min in a 10 μg/ml fibronectin solution and a single subsequent wash with PBS. The cells were plated at densities of 15000 cells per coverslip and were used for up to three days after plating, while the cell density was still low. All measurements were done in a Ringer solution containing (in mM) 135 NaCl, 5.4 KCl, 1.0 MgCl₂, 1.8 CaCl₂ and 5.0 Hepes, adjusted to pH 7.2 with NaOH.

### Staining

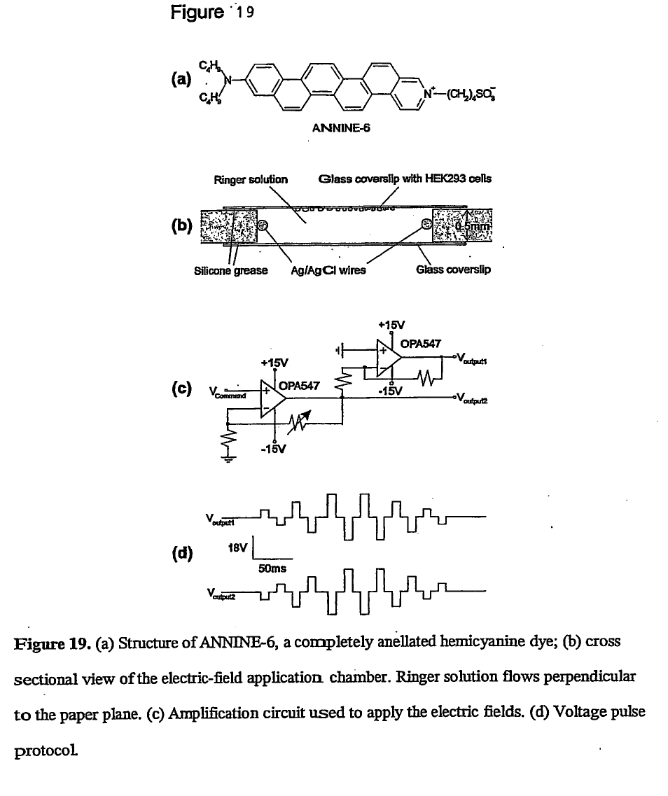

ANNINE-6 (FIG. 19a) was dissolved in a solution of 20% Pluronic F-127 DMSO (P-3000, Molecular Probes, Eugene, Oreg.) at a concentration of 0.5 mg/ml and sonicated for 15 min. The glass coverslip with the HEK293 cells was taken out of the culture well, exposed to the staining solution (dye stock solution diluted 1:100 into Ringer) for 5 min and finally washed with pure Ringer solution.

### Electric-Field Application

To change the voltage across the cell membrane we applied an extracellular electric field. This method (Gross et al., 1986) has a somewhat reduced voltage calibration accuracy but is much simpler than whole-cell recordings, for example, permitting the transfer of the recording chamber between microscope setups thereby allowing the exploration of a wider parameter range. Our custom-made chamber (FIG. 19b) consists of a 2.6 mm wide, and 30 mm long slit in a 0.5 mm thick PMMA (Plexiglas) plate. Two chlorided silver wires (diameter 0.127 mm, Cat. No. 7860, A-M Systems, INC., Everett, Wash.) for the application of the electrical field were fixed to the long edges of the channel with two-component epoxy resin glue (plus sofortfest, UHU, Bühl, Germany). This chamber was sealed on the bottom with a clean coverslip (24 mm×50 mm) attached with silicone grease (Bayer-silicone, Bayer AG, Leverkusen, Germany). The coverslip with the stained HEK293 cells was dried around the edges to get a watertight seal with silicone grease and then mounted cells down on the top side of the chamber. Two openings allowed the continuous perfusion (about 2 ml/min) with physiological saline using a gravity-fed perfusion system.

The voltage stimuli were generated by an arbitrary waveform generator (model 395, wavetek, Ismaning, Germany) and amplified by a custom-built push-pull circuit (using two operational amplifiers, OPA547, Texas Instruments Inc., Texas, USA, FIG. 19c). To avoid electrolysis at the electrodes and ionophoresis of cell-surface components all applied voltage waveforms were purely AC. Short pulses were used to minimize heating (FIG. 19d). The application of the pulse protocol was synchronized to the start of line scan acquisition.

Whole-cell tight-seal voltage-clamp recordings on HEK293 cells were preformed using pipettes with a resistance of around 6 MOhm and an epc9 amplifier (HEKA, Göttingen, Germany).

### Imaging

For one-photon excitation we used laser scanning microscopes (Zeiss LSM 510 NLO and Leica TCS SP2) with Ar-ion laser excitation (458 nm, 488 nm, or 514 nm). We used laser illumination instead of, for example, Xe arc-lamps because narrow spectral width and high intensities are needed to obtain sufficient excitation of the dye at the spectral edge. The excitation light at the Zeiss microscope was coupled in via the main dichroic beam splitters (HFT 458/514, reflecting the 458 nm and 514 nm laser lines or HFT KP 700/488, a combined 700 nm short pass and 488 nm reflector). The emitted light was filtered by a long-pass filter (LP560). The Leica microscope uses an acousto-optical main beam splitter and fluorescence was collected in the range of 520 nm to 800 nm. The maximal excitation intensities at the sample were for the Zeiss (Leica) microscope: 1.3 mW (1.5 mW) at 514 nm, 200 μW (250 μW) at 488 nm and 28 μW (35 μW) at 458 nm. The pinhole was completely opened up (8.5 airy units) to collect as much light as possible. A Zeiss IR 40×/0.8 water immersion objective was used at the Zeiss microscope and a Leica HCX PL APO CS 63×/1.32 oil immersion objective at the Leica.

For the two-photon experiments we initially used the Zeiss LSM 510 NLO microscope (beam splitter: HFT KP 700/488; filter: LP545) coupled to a Ti:Sapphire laser (Mira 900, Coherent) and later a custom-built setup that was coupled to a Ti:Sapphire laser (Mira 900F, Coherent) equipped with special cavity mirrors that allowed tuning to wavelengths of up to 1050 nm. In the custom microscope the fluorescence was filtered first with an IR-blocking heat mirror (Calflex-X, Linos AG, Göttingen, Germany) then a long-pass filter LP530 (AHF analysentechnik, Tübingen, Germany). The approximate average powers of the mode-locked laser at the sample were: 7.5 mW for 1040 nm, 5 mW for 1000 nm, and 4 mW for 960 nm. For all 2-photon measurements we used a Zeiss IR 40×/0.8 water immersion objective. In the custom 2PM setup fluorescence light was detected using GaAsP photo-multipliers (H7422P-40mod, Hamamatsu).

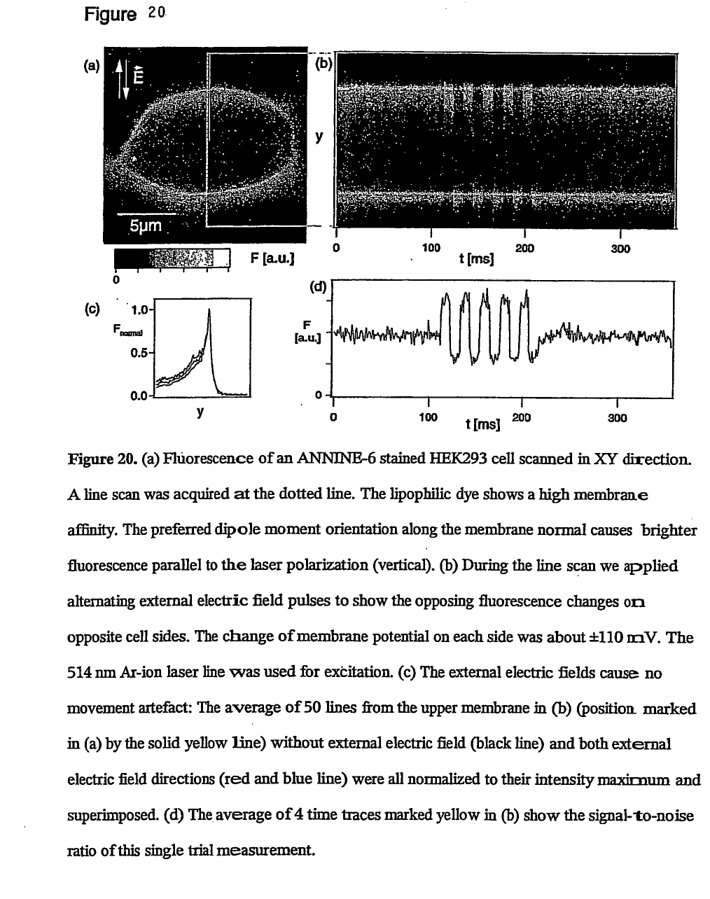

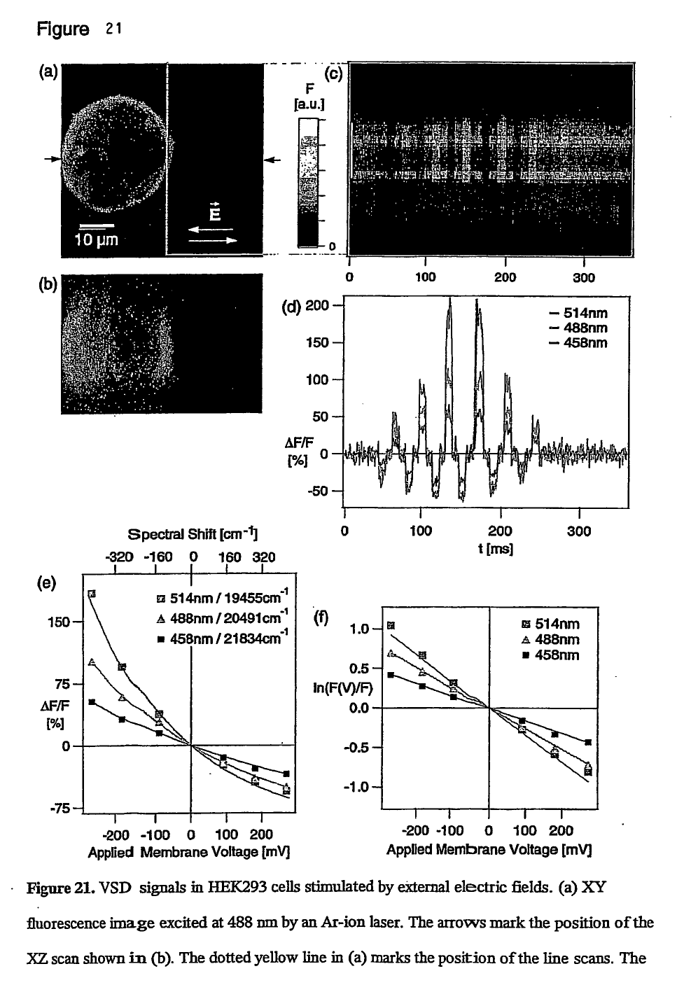

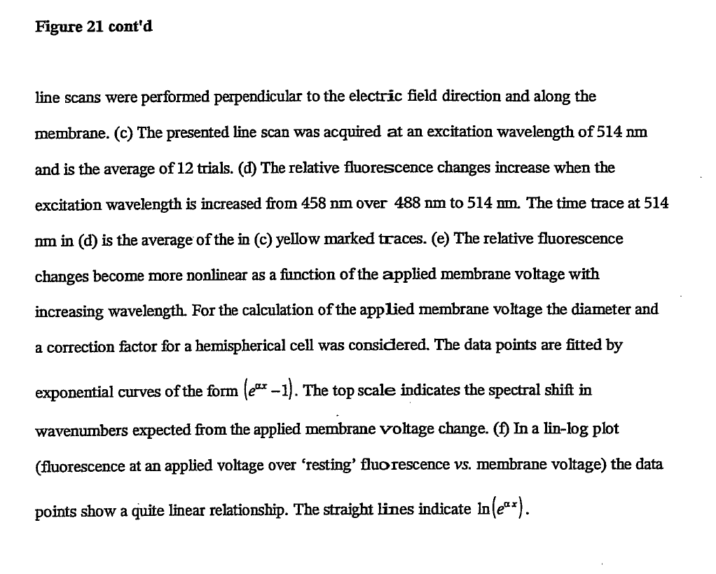

Line scans were oriented across (FIG. 20) or tangential to (FIGS. 21,22) the cell studied. Care was always taken to orient the polarization of the excitation light perpendicular to the membrane to ensure alignment with the absorption dipole of the dye. The temporal resolution was about 1 ms (0.96 ms per line for the Zeiss microscope and 1.00 ms for both the Leica microscope and for the custom 2PM). During the line scan we applied voltage pulses (10 ms duration spaced by 10 ms pauses) with opposite polarities to the silver-wires, at first in an increasing then in a decreasing order (FIG. 19d). The total voltages were 12, 24, and 36 V, which, given a wire spacing of 2.4 mm, correspond to electric field strengths of 5 mV/μm, 10 mV/μm and 15 mV/μm. After collecting data at the different excitation wavelengths 3D stacks of the cells were taken. All measurements were shot-noise limited. This was explicitly confirmed for the Zeiss and the custom 2PMs by the linear dependence of the variance on the mean fluorescence intensity.

### Analysis

All line scans were background corrected by subtraction of a value resulting from an averaged time trace outside the cell. We didn't correct for bleaching, which was almost imperceptible. Multiple experiments on the same cell and with the same excitation wavelength were usually averaged before calculating relative changes in fluorescence (□F/F). As resting fluorescence (F) we used the mean of the 20 data points before the beginning of the voltage stimulus. To get □F values we averaged the central 8 data points during the pulse, which is 10 points long, and then subtracted the mean of the values (each calculated correspondingly) for the preceding and following interpulse intervals. We only considered measurements from cells where fluorescence responses were still present after measurements with a complete wavelength set had been performed and the 3-D stack was taken.

### Results

### One-Photon Excitation

To validate the trans-cellular field simulation method, we first performed responsivity measurements using wavelength (458 nm, 488 nm) for which the sensitivity has been published (Kuhn and Fromherz, 2003). We find reasonable agreement (488 nm: −0.28%/mV in HEK293 compared to −0.29%/mV in Leech). We then proceeded to use a wavelength (514 nm) more towards the long edge of the absorption spectrum to test whether the sensitivity increases further towards longer wavelengths. To rule out spurious signals that might be created as the result of movement, for example caused by external electric field-induced ionophoresis or heating, we scanned through cells parallel to the applied field and perpendicular to the cell membrane (FIG. 20). No lateral movement was seen of the cell is membrane on either side of the cells, as is evident from the straightness of the bright lines in the line scan (FIG. 20b) and can be more closely examined by looking at the normalized plots of intensities along the scan line averaged over the 0, −110 mV and +110 mV stretches of the line scan (FIG. 20c). Here, as in all experiments, the laser polarization was aligned with the absorption dipole of the dye, which has for comparable dyes been measured to be oriented largely perpendicular to the membrane (Lambacher and Fromherz, 2001; Visser et al., 1995). Further evidence for the orientation of the absorption dipole is the variation of the fluorescence intensity around the cell (FIG. 20a) with minima at the 3 and 9 o'clock positions, where the polarization is parallel to the membrane. As expected with trans-field stimulation, the electric field on opposite sides of the cell points at the same absolute direction but in opposite direction relative to the dye molecules in the outer leaflet of the membrane bilayer. The signals, therefore, have different directions on opposite sides of a cell (FIG. 20b). The voltage resolution in single-trial measurements with 1 ms temporal resolution was about 12 mV rms (FIG. 20d).

To increase the number of scan-line pixels on the membrane, all further measurements were performed with the scan line tangential to the cell. The laser polarization, of course, was kept parallel to the electric field (FIG. 21). The image of a typical cell is shown in FIG. 3a. A XZ scan (FIG. 21b) reveals that only little fluorescence is generated inside the cell. One problem when using trans-cellular stimulation is that the actual voltage drop across the membrane depends on the cellular geometry and the estimation of precise values for the voltage drop across a particular membrane would require detailed electrostatic calculations, even if the cytosol can be assumed to be isopotential. Since we were mainly interested in the wavelength dependence of the responsivity we used an approximation to calculate the membrane voltage (Gross et al., 1986). For spherical cells in free solution or for hemispherical cells sitting on a coverslip the actual transmembrane voltage is larger by a factor of 1.5 (Gross et al., 1986) than the voltage drop expected by merely taking the product of cell diameter and field strength, since the insulating cell body displaces the current flow. We also assumed the voltage drop to be equally divided between both membranes, which was confirmed for the cell in FIG. 2 by the complementary fluorescence changes seen on opposite sides of the cell. The peak membrane voltage change for the cell of FIG. 21, which has an extent of 24 μm in X direction, was thus estimated at +90 mV for a field of ±5 mV/μm. We consider a membrane depolarizing voltage as positive change, which results in a fluorescence decrease if the outer leaflet is stained. Alternating dimming and brightening of the fluorescence intensity can clearly be seen in the averaged line scan data (FIG. 21c). FIG. 21d, where we compare the fluorescence changes for different excitation wavelengths (458 nm, 488 nm, and 514 nm), clearly demonstrates the increase of the responses towards longer wavelengths. As expected, the fluorescence followed the applied voltage instantaneously. Relative fluorescence changes are plotted as a function of membrane voltage in FIGS. 21e and 21f. Because ANNINE-6 is a pure Stark-shift probe, it is also possible to translate the membrane voltage change directly into a spectral shift (FIG. 21e, top scale). The responses become increasingly nonlinear at longer excitation wavelengths, deviating at 514 nm from linearity by factors of about 0.66 and 1.5 at +180 mV and −180 mV, respectively. If the excitation spectrum follows an exponential function (at least locally), the voltage dependence can be approximately linearized by using the logarithm of F(V)/F(V=0), as is shown in FIG. 21f. The slope sensitivities (α) at the resting potential can be calculated either by fitting (e^{ax}−1) to the data in FIG. 21e or by using the line slopes in FIG. 21f. For the cell shown in FIG. 21a we find −0.16%/mV, −0.26%/mV and −0.37%/mV for wavelengths of 458 nm, 488 nm, and 514 nm, respectively. The mean sensitivities (four cells measured) at these wavelengths are −0.16±0.02%/mV, −0.28±0.03%/mV, and −0.35±0.03%/mV, respectively.

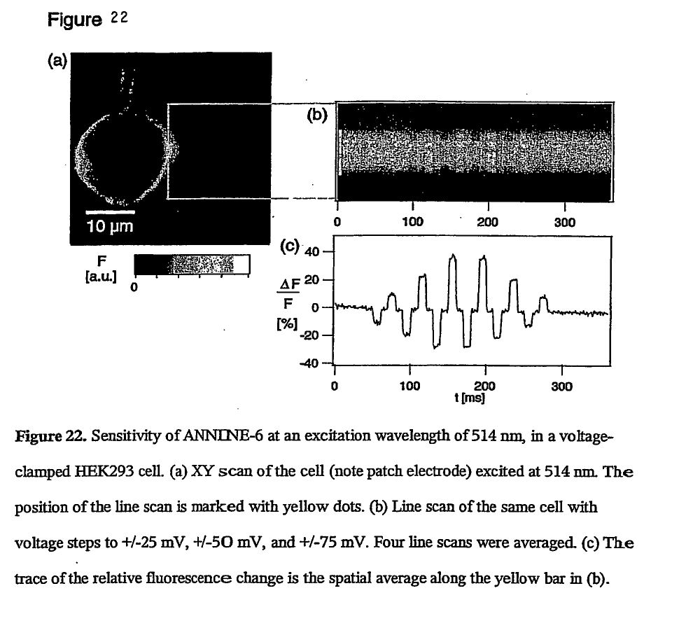

To test our assumption about the extracellular stimulation the sensitivity at 514 nm was tested by controlling the membrane voltage directly using tight-seal whole-cell recordings of HEK293 cells (FIG. 22). The mean sensitivity measured in four cells using voltage jumps of +/−25, +/−50 and +/−75 mV is −0.37±0.02%/mV, in agreement with the results achieved by the external electric field method.

### Two-Photon Excitation

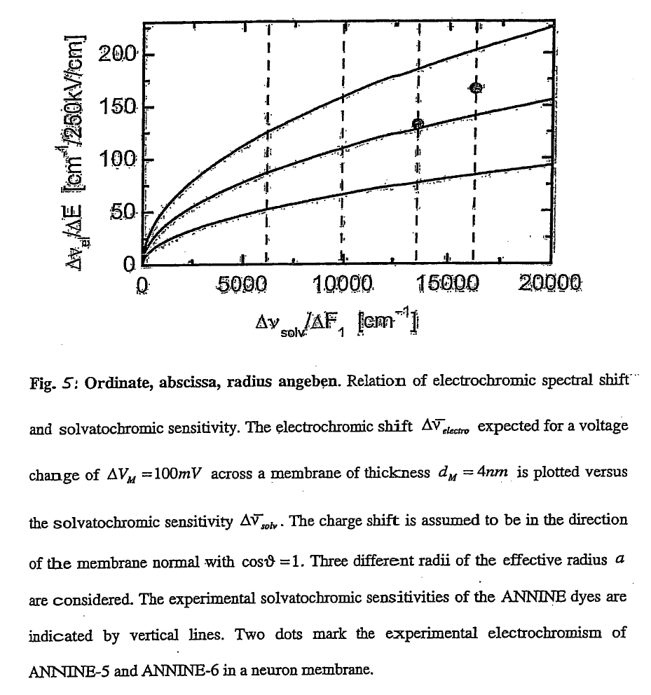

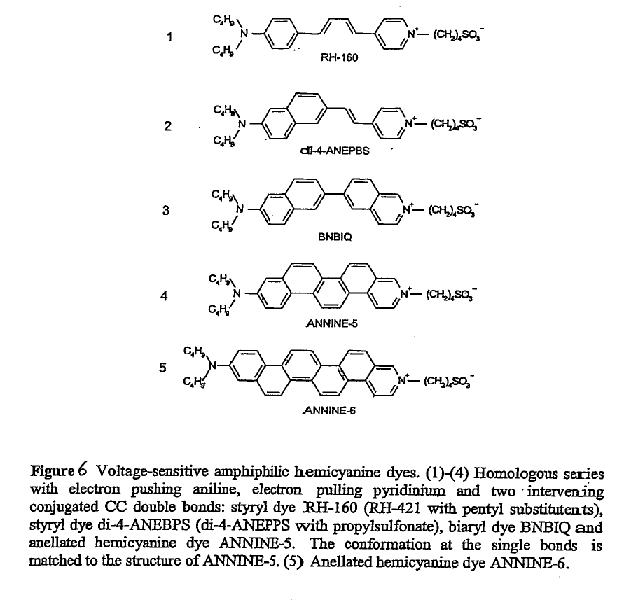

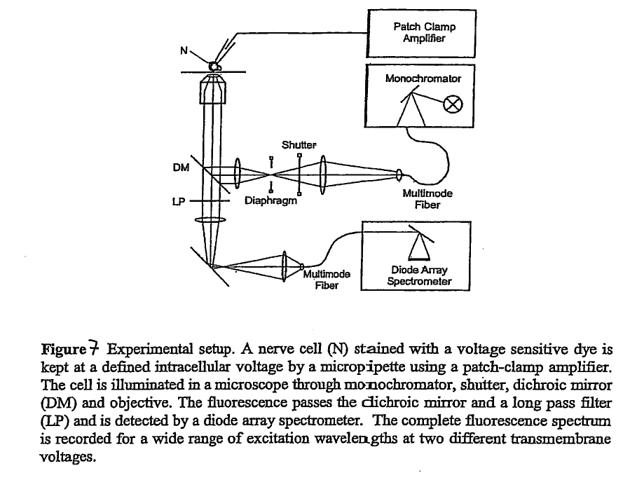

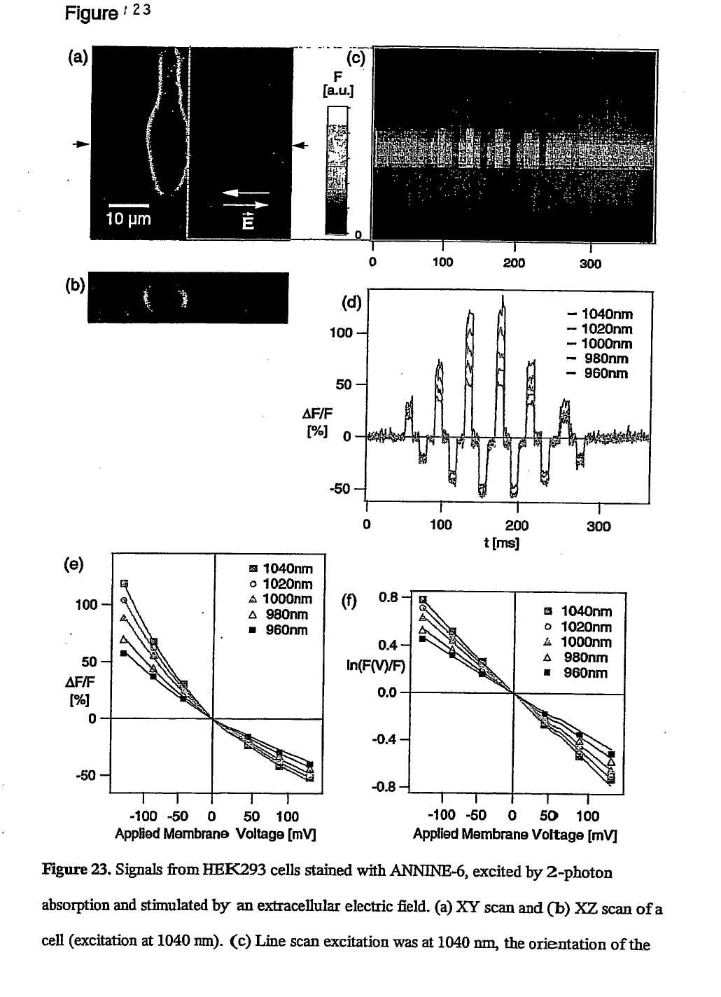

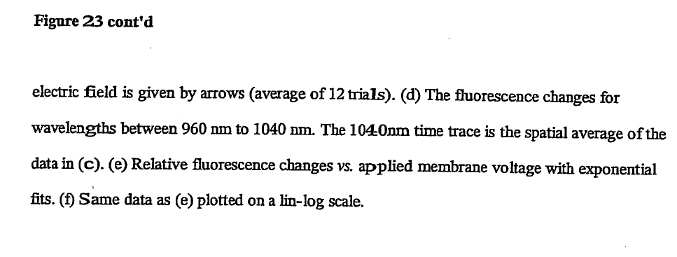

We proceeded to measure the voltage sensitivities under 2-photon excitation. Images show very low intracellular background (FIGS. 23a, b) with the reduction compared to the ‘confocal’ microscope (with an open pinhole used in FIG. 21), likely due to better out-of-focus discrimination. The long wavelength edge of the two-photon excitation spectrum is expected to resemble that of the one-photon spectrum because in molecules with a large change in dipole moment, the lowest one-photon transition is also strongly two-photon allowed (Dick and Hohlneicher, 1982). Initial experiments at 976 nm showed that with 2PE the sensitivity was slightly higher (by about 20%) than with 1PE at half the wavelength (488 nm), measured in the same cell. To confirm the increase towards the very edge of the spectrum we performed measurements at 960 nm, 980 nm, 1000 nm, 1020 nm, and 1040 nm (FIG. 23). As for 1PE the voltage dependence of the fluorescence signals became more sensitive and nonlinear with increasing excitation wavelength (FIG. 23e). The slope sensitivities for this cell at the resting potential were −0.35%/mV, −0.41%/mV, −0.49%/mV, −0.54%/mV and −0.59%/mV at 960 nm, 980 nm, 1000 nm, 1020 nm, and 1040 nm, respectively (FIGS. 23e, f). The mean sensitivities (4 cells) were −0.29±0.05%/mV, −0.35±0.05%/mV, −0.43±0.05%/mV, −0.47±0.06%/mV and −0.52±0.06%/mV, respectively. To test whether the rather high excitation intensities needed to excite sufficient fluorescence so far off the peak do damage the cells more than excitation at the peak of absorption, where the intensities required to get the same amount of fluorescence are much smaller, we exposed the same location in a single cell to hundreds of line scans. The cell, shown in FIG. 5, responded with undiminished amplitude (begin: −0.59±0.01%/mV, end: −0.58±0.02%/mV) after 900 line scans had been performed (with excitation at 1040 nm) over a period of over 2 h. These line scans represent about 350,000 individual voltage measurements each with an rms resolution of about 25 mV.

### Discussion

We have shown that when excited near the long wavelength edge of the absorption spectrum Stark-shift VSDs can show very large fractional sensitivities. This was specifically tested using ANNINE-6, which is one of a series of improved derivatives of the earlier voltage-sensitive hemicyanine dyes (Ephardt and Fromherz, 1993; Fluhler et al., 1985; Grinvald et al., 1983) that are particularly well suited for this method due to their large charge shift.

### Sensitivities in Different Cell Types, Comparison of 1-Photon and 2-Photon Excitation

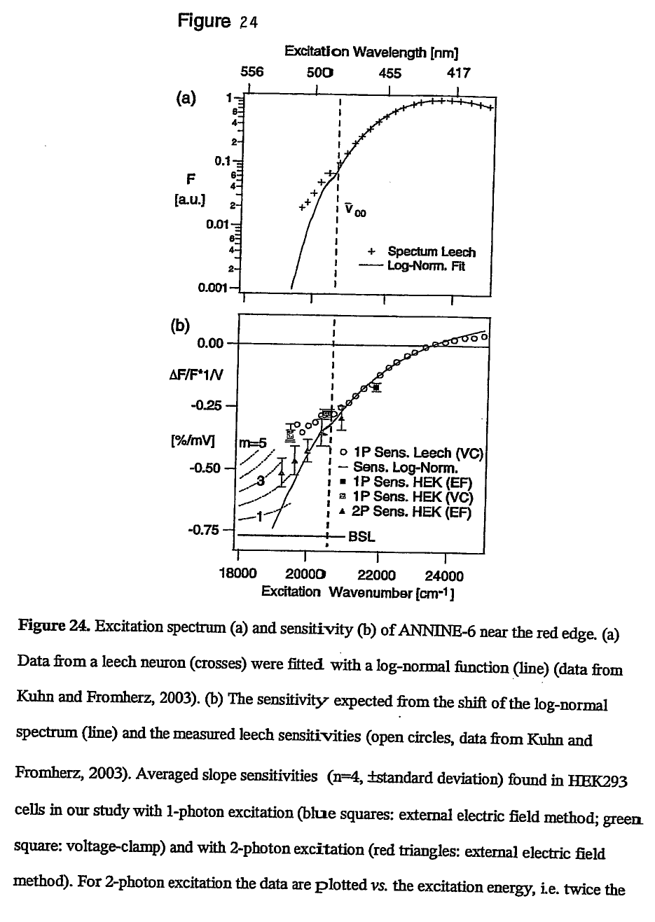

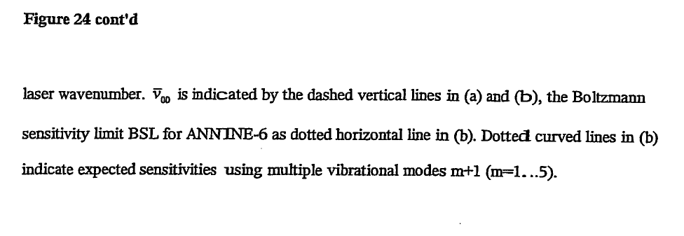

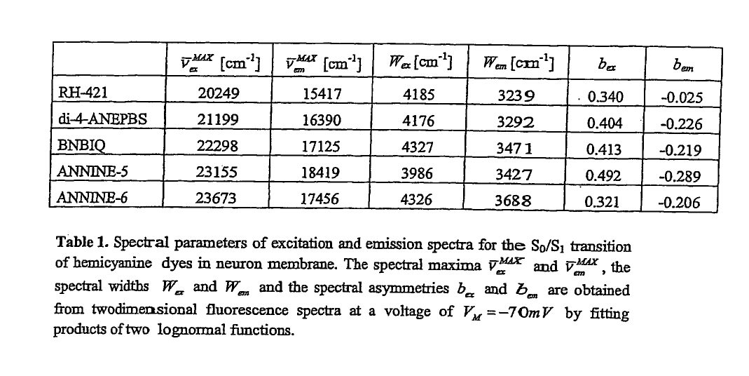

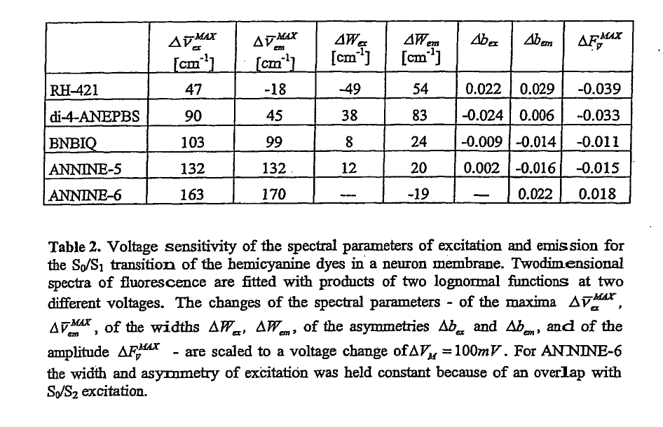

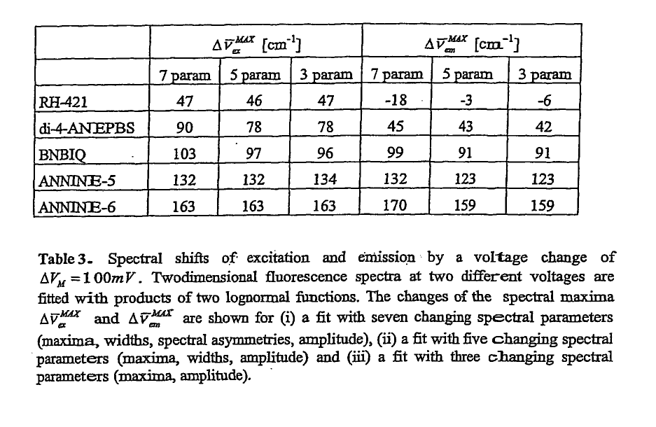

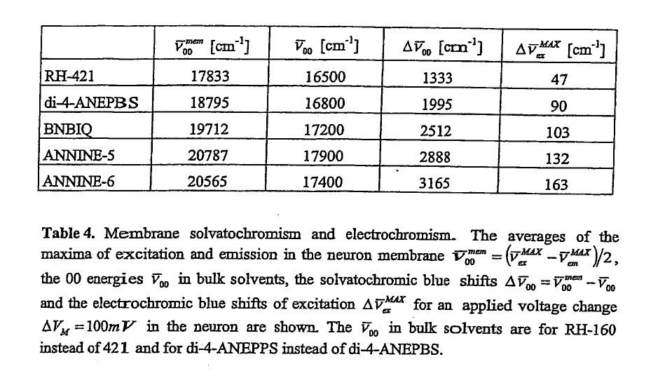

Because the voltage sensitivities of many VSDs are strongly cell-type dependent (Loew et al., 1992; Ross and Reichardt, 1979; Spors and Grinvald, 2002) we compared the sensitivities for ANNINE-6 in Retzius cells of the leech, measured by Kuhn and Fromherz, 2003, with those we found in HEK293 cells (FIG. 24b). Lack of cell-type dependence is desirable, since it allows calibrated voltage recordings even in unknown cells and reparations, and was expected from the pure Stark-effect character (Kuhn and Fromherz, 2003) of the ANNINE dye. At wavelengths of 458 nm and 488 nm the sensitivities agree within the measurement uncertainty. Even for 514 nm, where the measurements in leech neurons where thought to be flawed due to low fluorescence intensity and closeness to the beam splitter edge (Kuhn and Fromherz, 2003, and Kuhn and Fromherz, personal communication) both sensitivity values agree. The agreement of sensitivities taken with unpolarized light (Kuhn and Fromherz, 2003) and our measurements (with polarized laser excitation) also suggests that there are no significant sensitivity improvements with polarized excitation (this may be different in the two-photon case, see below). The main part of the spectrum and the calculated sensitivities are well fit by a log-normal function (Kuhn and Fromherz, 2003; Siano and Metzler, 1969). However, for the longer wavelengths (>480 nm, <21000 cm⁻¹) we find that the sensitivities both in leech and HEK293 cells start to deviate (FIG. 24) from those expected for the log-normal function (fitted globally). The two-photon sensitivities show the same trend. However, the measured sensitivities are generally larger with 2-photon excitation than with 1-PE. In the one case, where the 1P and 2P sensitivities were measured for the same cell for wavelengths of 488 nm and 976 nm, respectively, the 2P values were about 20% higher. The 2-photon sensitivities may, in fact, more closely reflect the actual “molecular” sensitivities due to the lower background, better optical sectioning (open pinhole with 1-PE), and better orientational selectivity of 2-photon excitation (Lakowicz et al., 1992). The nonlinearity of the sensitivity curves at the spectral edge reflects the curvature of the spectrum at the excitation wavelength.

### Sensitivity of VSD Recordings

Since fluorescence measurements in living tissue in general and VSD measurements in particular are always limited by photodamage it is crucial to carefully explore the optimization of excitation and detection spectral ranges. Optimization can be performed as described for the general case (Kuhn and Fromherz, 2003). In the absence of background fluorescence and if there are no limits on the excitation (both in terms of spectra and power density) rather straightforward, simplified rules can be established.

Since the generation of fluorescence photons requires molecular excitation, which is also the source of photobleaching and photodamage, it is crucial to maximize the information gained per molecular excitation, i.e. per generated (not detected) fluorescence photon. This distinction between generated and detected photons is important since large fractional intensity changes can sometimes be achieved by detecting only in a small wavelength band, at the expense of discarding a large fraction of the total fluorescence generated. Let us first assume that the excitation is by a collection of wavelengths. The response function is then the sum of response functions for each of the excitation wavelengths and, obviously, shifting excitation energy to wavelengths with better response will in crease the total SNR. An optimal value will be reached when all excitation occurs for the wavelength with the best response. This shows that monochromatic illumination at the appropriate wavelength is optimal, i.e. at least as good as illumination with any collection of wavelengths. To get simpler expressions we henceforth use an energy-proportional scale, i.e. wavenumbers. For a pure Stark-shift probe, for which the wavenumber dependent spectra can be written as ∈( ν̅ₑₓ,V)=∈₀( ν̅ₑₓ+κV) and f( ν̅ₑₓ,V)=f₀( ν̅ₑₓ+κV), the fractional change S_{V} in fluorescence as a function of excitation ν̅ₑₓ and emission ν̅ₑₘ wavenumbers is then (Kuhn and Fromherz, 2003)

$${S}_{V} ( {\overline{v}}_{ex} , {\overline{v}}_{em} ) = κ ( \frac{1}{ɛ ( {\overline{v}}_{ex} )} \frac{d ɛ ( {\overline{v}}_{ex} )}{d {\overline{v}}_{ex}} + \frac{1}{f ( {\overline{v}}_{em} )} \frac{d f ( {\overline{v}}_{em} )}{d {\overline{v}}_{en}} ) . \qquad ( 1 )$$

The dye specific constant κ is given by the ratio of spectral shift d ν̅_{ex,em} per membrane voltage change dV, ∈( ν̅ₑₓ) is the absorption cross section and f( ν̅ₑₘ) is the quantum spectrum of fluorescence. To optimally extract information from this spectrum we would have to detect each emission wavenumber separately and form a properly weighted average (Kuhn and Fromherz, 2003) since the voltage information that a photon provides depends on its wavenumber. The voltage sensitivity (Eqn. 1) consists of 2 terms, one term that results from the voltage dependence of the excitation spectrum and the other one from the voltage dependence of the emission spectrum. For excitation at the spectral edge however, this function is dominated by the excitation sensitivity as |∈′( ν̅ₑₓ)/∈( ν̅ₑₓ)| strongly increases towards the edge. Almost all emitted photons, independent of their wavenumber, yield a voltage signal with the same sign and with similar magnitude for the majority of the light. This is rather convenient since it makes the voltage variance almost independent of the emission wavelength for most of the emitted photons (except at for a small fraction at the very edge of the emission spectrum) and thus allows combining photons with equal weight, allowing near optimal detection with a single detector. The pure excitation voltage sensitivity [symbol] (S_{V}( ν̅ₑₓ) [symbol] can now be expressed as

$${S}_{V} ( {\overline{v}}_{ex} ) = κ \frac{1}{ɛ ( {\overline{v}}_{ex} )} \frac{d ɛ ( {\overline{v}}_{ex} )}{d {\overline{v}}_{ex}} . \qquad ( 2 )$$

An important parameter is the number of photons n needed to detect a certain voltage change ΔV. With shot-noise limited detection the voltage-induced fluorescence change [symbol] S_{V}( ν̅ₑₓ) [symbol] ΔV has to overcome the relative noise signal N given by

$$N = \frac{\sqrt{n}}{n} . \qquad ( 3 )$$

So the number of photons necessary to detect a given fluorescence change can be calculated by

$${S}_{V} ( {\overline{v}}_{ex} ) Δ V \geq \frac{1}{\sqrt{n}} . \qquad ( 4 )$$

In the case of ANNINE-6 the voltage sensitivity for excitation at, for example, 458 nm is −0.17%/mV which means that a voltage change of 10 mV could be detected with 3500 photons while 350 000 detected photons allow a voltage estimate with a standard deviation of 1 mV. With an excitation wavelength of 514 nm and a sensitivity of −0.35%/mV the number of photons to detect voltage changes of 10 mV and 1 mV are 820 and 82000, respectively.

FIG. 6b shows the sensitivity [symbol] S_{V}( ν̅ₑₓ) [symbol] as a function of excitation wavelength. To obtain a given accuracy one has to detect a certain number of photons. Therefore, the sensitivity is a direct measure for the number of voltage measurements of a given sensitivity one can make with a fixed number of photons before, for example, the sample bleaches or is photo-damaged. This illustrates the advantage gained from excitation at the spectral edge, provided, of course, the damage per molecular excitation is wavelength independent. This is an issue that will have to be explored further.

### Maximal Theoretical Sensitivity and Explanation of Tail Sensitivity

A common approximation used for spectral shapes is the log-normal function (Siano and Metzler, 1969), which would imply an ever increasing, in fact, diverging sensitivity toward longer wavelengths and the vanishing of excitation above a wavelength of 591 nm. However, simple considerations predict a tail in the absorption spectrum F that follows a Boltzmann distribution (Hinshelwood, 1940)

$$F = {F}_{0} {e}^{- \frac{E}{{k}_{B} T}} , \qquad ( 5 )$$

because an electronic transition between the ground and the excited state with the energy difference h ν̅₀₀c (h Planck constant, ν̅₀₀ wavenumber of the 00 transition, c light velocity) and an excitation energy h ν̅c ( ν̅ wavenumber of excitation), which is assumed to be below h ν̅₀₀c, requires additional vibrational energy E=h ν̅₀₀c−h ν̅c to reach the lowest excited state, in analogy to anti-Stokes lines in scattering theory. The absorption F is then proportional to the fraction of molecules with higher vibrational energy than E. Since for ANNINE-6 ν̅₀₀ is 20565 cm⁻¹ (486 nm) (Kuhn and Fromherz, 2003) most of the wavelengths we used here are, in fact, in the spectral tail. For a pure Stark-effect dye, implying a mere shift in the spectrum, the excitation efficiency then changes for small changes in the voltage ΔV by

$${( \frac{Δ F}{F} )}_{Max} = {e}^{- \frac{Δ μ Δ V cos θ}{d} - \frac{1}{{k}_{B} T}} - 1 = {e}^{- \frac{Δ E}{{k}_{B} T}} - 1 {\approx }^{Δ E < {k}_{B} T} - \frac{Δ E}{{k}_{B} T} \qquad ( 6 )$$

for excitation in the Boltzmann tail (Δμ is the intramolecular charge displacement upon electronic excitation, d is the membrane thickness, θ is the angle between charge displacement and membrane normal). This is the maximal relative change possible for a given change in charge shift (change in dipole moment) at a certain temperature. For ANNINE-6 the shift is 1.6 cm⁻¹/mV (Kuhn and Fromherz, 2003), which translates into a Boltzmann-tail sensitivity of −0.77%/mV. For comparison: the highest measured sensitivity value was −0.52±0.05%/mV (with 2-photon excitation at 1040 nm, corresponding to 520 nm). Because this sensitivity limit depends only on the shift of the excitation spectrum the expected sensitivity limit for other dyes should be lower (ANNINE-5: 1.3 cm⁻¹/mV; BNBIQ: 1.0 cm⁻¹/mV; di-4-ANEPBS: 0.9 cm⁻¹/mV; RH-421: 0.5 cm⁻¹/mV; Kuhn and Fromherz, 2003).

A more realistic description of the spectral sensitivity predicts a flattening of the spectral tail compared to the Boltzmann curve as more vibrational modes (m+1) are taken into account (Hinshelwood, 1940)

$$F = {F}_{0} {e}^{- \frac{E}{{k}_{B} T}} {\sum }_{p = 0}^{m} \frac{{( E / {k}_{B} T )}^{p}}{p !} . \qquad ( 7 )$$

This approach was, for example, used successfully to describe the long wavelength spectral tail of visual pigments (Lewis, 1955; Voss and Van Norren, 1984). FIG. 23b includes curves for m=1 to 5 and suggests that 3 or 4 vibrational modes are relevant for ANNINE-6. To really test, however, whether the multiple levels or residual background are responsible for the deviation from the log-normal behaviour measurements at even longer wavelengths would be necessary.

In practice a major limitation is the availability of sufficiently intense light sources since, as this paper shows, the optimal excitation wavelengths will almost always lie in a low absorption part of the spectrum. Furthermore, the range over which the excitation wavelength is nearly optimal, is typically narrow and does usually not coincide with a strong spectral line. Therefore, one has to resort to using a narrow spectral band and hence only a small fraction of the total light output from a broad spectrum light source such as an arc or an incandescent bulb. The use of lasers, which can provide narrow-band excitation, for wide-field illumination is beset by speckle problems and often a line at the appropriate wavelength is not available. A further problem can be the excitation of tissue auto fluorescence as increasingly intense light is used for excitation.

While we have only tested extreme red excitation on one dye (ANNINE-6) it should be applicable to all spectral shift dyes with a sufficiently steep long-wavelength spectral fall-off.

### REFERENCES FOR EXAMPLE 5

Denk W. Strickler J H, Webb W W. 1990. 2-Photon Laser Scanning Fluorescence Microscopy. Science 248(4951):73-76.
Dick B, Hohlneicher G. 1982. Importance of Initial and Final-States as Intermediate States in 2-Photon Spectroscopy of Polar-Molecules. J. Chem. Phys. 76(12):5755-5760.
Ephardt H, Fromherz P. 1993. Fluorescence of Amphiphilic Hemicyanine Dyes without Free Double-Bonds. J. Phys. Chem. 97(17):4540-4547.
Fluhler E, Burnham V G, Loew L M. 1985. Spectra, membrane binding, and potentiometric responses of new charge shift probes. Biochem. 24 (21):5749-55.
Grinvald A, Fine A, Farber I C, Hildesheim R. 1983. Fluorescence monitoring of electrical responses from small neurons and their processes. Biophys. J. 42(2):195-8.
Grinvald A, Frostig R D, Lieke E, Hildesheim R. 1988. Optical Imaging of Neuronal-Activity. Physiol. Rev. 68(4):1285-1366.
Gross D, Loew L M, Webb W W. 1986. Optical imaging of cell membrane potential changes induced by applied electric fields. Biophys. J. 50(2): 339-48.
Hinshelwood C N. 1940. The kinetics of chemical change. Oxford: Clarendon Press.
Hübener G. Lambacher A, Fromherz P. 2003. Anellated hemicyanine dyes with large symmetrical solvatochromism of absorption and fluorescence. J. Phys. Chem. B 107(31):7896-7902.
Kuhn B, Fromherz P. 2003. Anellated hemicyanine dyes in a neuron membrane: Molecular Stark effect and optical voltage recording. J. Phys. Chem. B 107(31):7903-7913.
Lakowicz J R, Gryczynski I, Gryczynski Z, Danielsen E, Wirth M J. 1992. Time-Resolved Fluorescence Intensity and Anisotropy Decays of 2,5-Diphenyloxazole by 2-Photon Excitation and Frequency-Domain Fluorometry. J. Phys. Chem. 96(7):3000-3006.
Lambacher A, Fromherz P. 2001. Orientation of hemicyanine dye in lipid membrane measured by fluorescence interferometry on a silicon chip. J. Phys. Chem. B 105(2):343-346.
Lewis P R. 1955. A theoretical interpretation of spectral sensitivity curves at long wavelength. J. Physiol.—London 130(1):45-52.
Loew L M. 1982. Design and characterization of electrochromic membrane probes. J. Biochem. Biophys. Meth. 6:243-260.
Loew L M, Cohen L B, Dix J, Fluhler E N, Montana V, Salama G, Wu J Y. 1992. A naphthyl analog of the aminostyryl pyridinium class of potentiometric membrane dyes shows consistent sensitivity in a variety of tissue, cell, and model membrane preparations. J. Membr. Biol. 130(1):1-10.
London J A, Zecevic D, Cohen L B. 1987. Simultaneous optical recording of activity from many neurons during feeding in Navanax. J. Neurosci. 7 (3):649-61.
Meyer E, Muller C O, Fromherz P. 1997. Cable properties of dendrites in hippocampal neurons of the rat mapped by a voltage-sensitive dye. Eur. J. Neurosci. 9(4):778-85.
Ross W N, Reichardt L F. 1979. Species-specific effects on the optical signals of voltage-sensitive dyes. J. Membr. Biol. 48(4):343-56.
Salinas E, Sejnowski T J. 2001. Correlated neuronal activity and the flow of neural information. Nature Rev. Neurosci. 2(8):539-550.
Siano D B, Metzler D E. 1969. Band Shapes of Electronic Spectra of Complex Molecules. J. Chem. Phys. 51(5):1856-&.
Singer W. 1999. Neuronal synchrony: A versatile code for the definition of relations? Neuron 24(1):49-65.
Spors H, Grinvald A. 2002. Spatio-temporal dynamics of odor representations in the mammalian olfactory bulb. Neuron 34(2):301-15.
Visser N V, Vanhoek A, Visser A J W G, Frank J, Apell H J, Clarke R J. 1995. Time-Resolved Fluorescence Investigations of the Interaction of the Voltage-Sensitive Probe Rh421 with Lipid-Membranes and Proteins. Biochem. 34(37):11777-11784.
Voss J J, Van Norren W A. 1984. Limits of the visual spectrum. In: Doorn A J, Van de Grind W A, Koenderink J J, editors. Limits in perception. Utrecht: VNU Science Press. p 69-84.

1. A method of determining voltage changes by means of a voltage-sensitive dye, characterized in that the voltage-sensitive dye is irradiated with light having a wavelength at which the dye has an absorption ≦20% of its absorption maximum and fluorescence caused by irradiation with light is measured.

2. The method according to claim 1, characterized in that the wavelength of the irradiated light is such that the dye has an absorption of ≦12%, in particular ≦8% and preferably ≦2% of its absorption maximum at said wavelength.

3. The method according to claim 1, characterized in that the wavelength of the irradiated light is in the longer wavelength range, related to the absorption maximum.

4. The method according to claim 1, characterized in that an increase or decrease of the fluorescence is measured.

5. The method according to claim 1, characterized in that it is used to determine voltage changes in cells.

6. The method according to claim 1, characterized in that it is used to determine voltage changes in membranes, especially cell membranes.

7. The method according to claim 1, characterized in that, as voltage-sensitive dye, a compound of formula (I) 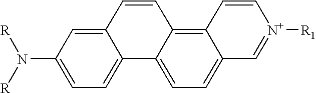
formula (II) 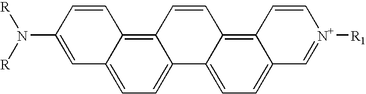
is used, wherein
each R independently is a hydrocarbon residue, which optionally can be substituted with hydroxyl,
R¹ is a monovalent residue,
n is an integer from 1 to 9, and
n is an integer from 0 to 8,
which compounds optionally can have one or more substituents at ring carbon atoms.

8. The method according to claim 1, characterized in that ANNINE-4, ANNINE-5, ANNINE-6, ANNINE-7, ANNINE-8 or ANNINE-9 is used as a voltage-sensitive dye.

9. The method according to claim 1, characterized in that a change of fluorescence radiation caused by the Stark effect is measured.

10. The method according to claim 1, characterized in that a two-photon excitation is effected.

11. Voltage-sensitive dye having the formula (I) 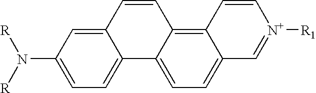
formula (II) 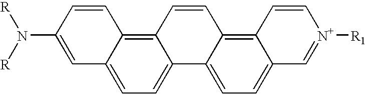
wherein
each R independently is a hydrocarbon residue, which optionally can be substituted with hydroxyl,
R¹ is a monovalent residue,
n is an integer from 1 to 9, and
n is an integer from 0 to 8,
which compounds optionally can have one or more substituents at ring carbon atoms.
